(19) United States
(12) Patent Application Publication
(10) Pub. No.: US 2009/0318804 A1
(43) Pub. Date: Dec. 24, 2009

(54) Cryotherapy Planning and Control System

(75) Inventors: Ofer Avital, Yokneam Ilit (IL); Eyal Kochivi, Haifa (IL); Amir Pansky, Atlit (IL); Yaron Hefetz, Herzlia (IL); Pazit Pianka, Kochav Yair (IL)

Correspondence Address: MARTIN D. MOYNIHAN d/b/a PRTSI, INC., P.O. BOX 16446, ARLINGTON, VA, 22215, US

(73) Assignee: Galil Medical Ltd., Yokneam (IL)

(21) Appl. No.: 12/226,900

(22) PCT Filed: May 2, 2007
(86) PCT No.: PCT/IL07/00537

§ 371 Date: Mar. 5, 2009

Related U.S. Application Data
(60) Provisional application No. 60/796,519, filed on May 2, 2006.

(51) Int. Cl. A61B 8/14; A61B 18/02

(52) U.S. Cl. 600/439; 600/445; 606/21

(57) ABSTRACT

The present invention relates to devices and methods for planning and supervising minimally invasive surgery. Included are enhancements to systems for planning, monitoring, and controlling cryosurgery.

### RELATED APPLICATIONS

The present application is related to U.S. patent application Ser. No. 11/219,648, filed on Sep. 7, 2005, which is a Continuation of U.S. patent application Ser. No. 11/066,294, filed on Feb. 28, 2005, which is a Divisional of U.S. patent application Ser. No. 09/917,811, filed on Jul. 31, 2001, now U.S. Pat. No. 6,905,492, issued on Jun. 14, 2005, which claims priority from U.S. Provisional Patent Application No. 60/221,891, filed on Jul. 31, 2000.

The present application further claims priority U.S. Provisional Patent Application 60/796,519, filed May 2, 2006. The contents of all of the above-mentioned applications are incorporated herein by reference.

This application is related to two other PCT applications being filed on even date with this application in the Israel Receiving Office having the titles and PROBE INSERTION GUIDE WITH USER-DIRECTING FEATURES and CRYOTHERAPY INSERTION SYSTEM AND METHOD, and Attorney docket Nos. 39261 and 39262, and sharing applicant Galil Medical Ltd. with this Application, the disclosures of which are incorporated herein by reference.

### FIELD AND BACKGROUND OF THE INVENTION

The present invention relates to systems and methods for planning and supervising ablative cryosurgery. More particularly, the present invention relates systems and methods for collecting information about a patient, planning a surgical intervention, and for executing the planned intervention successfully.

Schatzberger, in U.S. Pat. No. 6,142,991 teaches three dimensionally mapping an organ of a patient so as to form a three dimensional grid thereof, and applying a multi-probe system introducing probes into the organ according to the grid, so as to enable systematic high-resolution three dimensional cryosurgical treatment of the organ and selective destruction of the treated tissue with minimal damage to surrounding, healthy, tissues.

The Seednet Training And Planning Software (“STPS”) marketed by Galil Medical Ltd. of Yokneam, Israel provides a system for displaying, and allowing an operator to manipulate, a model of a prostate, and further allows an operator to plan a cryoablation intervention and to visualize the predicted effect of that planned intervention on the prostate tissues.

U.S. Pat. No. 6,905,492 to Zvuloini et al., and pending U.S. patent application Ser. No. 11/219,648, also by Zvuloni et al., which are is incorporated herein by reference, teach a system and method for planning a cryoablation procedure by simulating such a procedure based on preparatory imaging of a target site in a patient, by simulating the procedure, by recommending procedural steps and by evaluating procedural steps specified by a user. Zvuloni teaches use of integrated images displaying, in a common virtual space, a three-dimensional model of a surgical intervention site based on digitized preparatory images of the site from first imaging modalities, simulation images of cryoprobes used according to an operator-planned cryoablation procedure at the site, and real-time images provided by second imaging modalities during cryoablation. Zvuloni further teaches system-supplied recommendations for and evaluations of the planned cryoablation procedure, and system-supplied feedback to an operator and system-supplied guidance and control signals for operating a cryosurgery tool during cryoablation.

Additional patents and patent applications which provide background information relevant to the present invention include U.S. Pat. Nos. 6,139,544, 6,485,422, 6,544,176, 6,694,170, 6,206,832, 6423,009, 6,610,013, 5,531,742, 5,377,683, 4,672,963, U.S. Patent Applications 20020016540, 20020198518, 20020198518, and PCT Application WO04051409.

### SUMMARY OF THE INVENTION

The present invention relates in particular to improved technologies for pre-operative user-input characterization of surgical target sites, for pre-operative and operative use of ultrasound and other imaging modalities to characterize surgical sites before and during surgery and more particularly during cryosurgery, to production and display of predictive evaluations of planned and real-time situations in terms of probabilities of tissue survival, and to improved methods for relating planned surgical procedures to actual surgical contexts.

Methods of prior art fail to provide adequate means for visualizing therapeutic probes in operative situations under certain popular imaging modalities, and in particular fail to provide means for visualizing exact positions of cryoprobes before and most particularly during cryosurgery. Thus, there is a widely recognized need for, and it would be highly desirable to have, devices and methods for ascertaining exact positions of inserted therapeutic probes during cryosurgery when portions of body tissue are frozen. The present invention successfully addresses the shortcomings of the presently known configurations by providing means for doing so.

Methods of prior art fail to provide adequate means for visualizing surgical target environments under various standard clinical situations, such as for example during cryotherapy of a prostate under guidance of rectal ultrasound probe imaging. Thus, there is a widely recognized need for, and it would be highly desirable to have, devices and methods for visualization of an entire target locus during prostate surgery and in similar contexts. The present invention successfully addresses the shortcomings of the presently known configurations by providing means for doing so.

Methods of prior art fail to provide means for detailed control of contours of an ablation volume created by cooling of a given set of inserted cryoprobes. Yet, there is a widely recognized need to completely ablate certain lesions while protecting and preserving important anatomical structures in proximity of those lesions. The present invention successfully addresses the shortcomings of the presently known configurations by providing means for more accurately contouring borders of cryoablation volumes, thus helping to preserve healthy tissues in proximity to ablated lesions in a variety of contexts.

Methods of prior art provide surgical planning systems which fail to provide convenient means for adapting plans created with respect to pre-operative patient images to actual patient organ geographies once therapeutic probes have been inserted in a patient according to an initial plan. The present invention successfully addresses the shortcomings of the presently known configurations by providing means for doing so.

Methods of prior art provide for calculation and display of estimated surgical outcomes in terms of temperature gradients in tissues. Yet, there is a widely recognized need for, and it would be highly desirable to have, devices and methods for calculating and displaying therapy predictions in terms of assessed probabilities of tissue survival, as compared to user evaluations or automated evaluations of desirability of tissue survival. The present invention successfully addresses the shortcomings of the presently known configurations by providing means for inputting graduated tissue survival desirability scores for various tissues in a surgical context, and by providing means for calculating and displaying probabilities of tissues survival according in simulated or actual clinical contexts in a manner which facilitates comparing tissue survival desirability with tissue survival probability.

Surgical planning systems generally request user input in response to patient images created by imaging modalities such as CT scans, MRI and ultrasound images, in order to identify, differentiate and characterize tissues in the vicinity of a lesion. Thus, there is a widely recognized need for, and it would be highly desirable to have, devices and methods for facilitating the process of user evaluation of such images. The present invention successfully addresses the shortcomings of the presently known configurations by providing methods and devices for facilitating user input serving to characterize tissues presented in pre-operative patient imaging.

There is provided in accordance with an exemplary embodiment of the invention, an ultrasound system for use during surgery, comprising

(a) a first ultrasound probe;

(b) a second ultrasound probe;

(c) an image registration system operable to register, in a common coordinate system, information gleaned from operation of said first probe and information gleaned from operation of said second probe.

In an exemplary embodiment of the invention, the system comprises:

(d) an image display system operable to display an image which comprises information gleaned from said first probe and information gleaned from operation of said second probe.

In an exemplary embodiment of the invention, the system comprises a position sensor operable to report a position of at least one of said first and second ultrasound probes.

In an exemplary embodiment of the invention, the system comprises an echogenic probe insertable in a body and easily visible under ultrasound imaging.

In an exemplary embodiment of the invention, the system comprises a motorized probe positioner operable to respond to a positioning command by positioning at least one of said first and second ultrasound probes at a position designated by said command. Optionally, said probe positioner is operable to advance and retract said at least one probe within a body cavity. Alternatively or additionally, said probe positioner is operable to rotate said at least one probe around a longitudinal axis of said probe. Alternatively or additionally, said probe positioner is operable to impart both linear and rotational motions to said at least one probe.

In an exemplary embodiment of the invention, at least one of said first and second ultrasound probes is a probe sized for insertion into a body cavity. Optionally, said probe sized for insertion into a body cavity is a rectal probe. Alternatively or additionally, said probe sized for insertion into a body cavity is a vaginal probe.

In an exemplary embodiment of the invention, at least one of said first and second ultrasound probes is designed to be used while positioned externally to a body.

In an exemplary embodiment of the invention, one of said first and second ultrasound probes is a rectal probe, and another of said first and second ultrasound probes is operable to be used when positioned externally to a body.

There is provided in accordance with an exemplary embodiment of the invention, a method for ultrasound imaging of a target within a body of a patient, comprising:

(a) using a first ultrasound probe to image said target from a first direction and using a second ultrasound probe to image said target from a second direction; and

(b) displaying said first and said second images simultaneously to a user, thereby providing simultaneous images of said target from two different perspectives. Optionally, the method comprises comprising inserting in a vicinity of said target a probe so configured as to be easily visible under ultrasound imaging. Alternatively or additionally, the method comprises comprising inserting into a vicinity of said target a probe having a vibrator attachment operable to vibrate said probe, and wherein at least one of said ultrasound probes comprises a Doppler detector operable to detect vibration of said vibrating probe.

In an exemplary embodiment of the invention, the method comprises alternating operation of said first and second ultrasound probes, thereby avoiding signal interference between said first and second probes.

In an exemplary embodiment of the invention, said first ultrasound probe is positioned outside said body and said second ultrasound probe is inserted in a body cavity. Optionally, said second ultrasound probe is one of a group consisting of a rectal ultrasound probe and a vaginal ultrasound probe.

In an exemplary embodiment of the invention, the method comprises comprising operating a cryoprobe in a vicinity of said target during said imaging.

In an exemplary embodiment of the invention, said target is a prostate.

There is provided in accordance with an exemplary embodiment of the invention, a method for ultrasound imaging of a target within a body, comprising:

(a) using a first ultrasound probe in a first position to receive ultrasound echoes from said target and using a second ultrasound probe at a second position distant from said first position to receive ultrasound echoes from said target; and

(b) creating an image which comprises information received from said first ultrasound probe and information received from said second ultrasound probe. Optionally, the method comprises alternating operation of said first and second ultrasound probes, thereby avoiding acoustical interference between said first and second probes. Alternatively or additionally, the method comprises displaying said created image.

In an exemplary embodiment of the invention, said first ultrasound probe is positioned external to said body and said second ultrasound probe is inserted in a body cavity.

In an exemplary embodiment of the invention, said body cavity is a rectum.

In an exemplary embodiment of the invention, said body cavity is a vagina.

In an exemplary embodiment of the invention, the method comprises operating a cryoprobe in a vicinity of said target during said imaging.

In an exemplary embodiment of the invention, said target is a prostate.

There is provided in accordance with an exemplary embodiment of the invention, a method for monitoring a cryoablation operation, comprising:

(a) inserting a cryoprobe in a body of a patient and cooling said cryoprobe, forming an ice-ball;

(b) using a first ultrasound probe positioned at a first position to image said ice-ball from a first perspective; and

(c) using a second ultrasound probe positioned at a second position to image said ice-ball from a second perspective. Optionally, the method comprises simultaneously displaying a first image showing a view of said ice-ball from said first perspective and a second image showing a view of said ice-ball from said second perspective. Alternatively or additionally, the method comprises creating and displaying a composite image comprising information received from said first ultrasound probe and also comprising information received from said second cryoprobe.

In an exemplary embodiment of the invention, said first ultrasound probe is operated from outside a patient's body and said second ultrasound probe is inserted in a body cavity. Optionally, said second ultrasound probe is inserted in a rectum. Alternatively or additionally, said second ultrasound probe is inserted in a vagina.

In an exemplary embodiment of the invention, the method comprises utilizing a position sensor to sense and report position of at least one of said ultrasound probes.

There is provided in accordance with an exemplary embodiment of the invention, a system for cryoablation comprising:

(a) first and second cryoprobes, each operable to cool to cryoablation temperatures and also operable to heat;

(b) a cryogen control unit programmed to alternate between a first mode which comprises heating said first cryoprobe while cooling said second cryoprobe, and a second mode which comprises heating said second cryoprobe while cooling said first cryoprobe. Optionally, said cryogen control unit is programmed to supply heating gas to said first cryoprobe while supplying cooling gas to said second cryoprobe and to supply cooling gas to said first cryoprobe while supplying heating gas to said second cryoprobe.

There is provided in accordance with an exemplary embodiment of the invention, a method of cryoablation which comprises alternating a first mode which comprises cooling a first cryoprobe while heating a second cryoprobe with a second mode which comprises heating said first cryoprobe while cooling said second cryoprobe.

There is provided in accordance with an exemplary embodiment of the invention, a method of contouring an ablation volume comprising timing supply of cooling and heating gasses to a plurality of cryoprobes inserted in a body of a patient so as to effect anti-synchronized cooling of said cryoprobes, thereby creating an ablation volume with indented contour.

There is provided in accordance with an exemplary embodiment of the invention, a surgery apparatus comprising:

(a) a probe insertable into a body of a patient;

(b) a vibrator attachable to said probe, and operable to impart a vibration to said probe while said probe is inserted in a patient;

(c) an ultrasound system which comprises a Doppler detector operable to detect said vibrating probe by detecting Doppler variations in echoes received from said probe.

(d) an image registration system operable to register in a common coordinate system a plurality of ultrasound images generated from different perspectives by recognizing, within said images, probe echoes having same Doppler variations.

There is provided in accordance with an exemplary embodiment of the invention, a method for cryotreatment of an organ of a patient, comprising:

(a) using an imaging modality to produce a first image of a body portion;

(b) defining a treatment goal with respect to said first image;

(c) providing therapeutic probe positions for achieving said treatment goal;

(d) inserting therapeutic probes into a patient;

(e) using an imaging modality to produce a second images of said body portion;

(f) calculating probe operating parameters based on probe positions observable in said second image; and

(g) utilizing said inserted probes according to said calculated probe operating parameters to treat said patient.

In an exemplary embodiment of the invention, (c) comprises suggesting by a user. Alternatively or additionally, (c) comprises evaluation by a computerized system. Alternatively or additionally, (c) comprises evaluation by a computerized system and user acceptance or modification of said positions based on a predicted outcome of said evaluation. Optionally, said modification includes at least one of adding a probe, removing a probe and changing a probe location.

In an exemplary embodiment of the invention, said defining a treatment goal comprises displaying said first images to a user and receiving input from said user, said input serving to define a treatment goal.

In an exemplary embodiment of the invention, the method comprises inserting a position-marking probe visible under said imaging modality to mark a reference position in said body portion prior to production of said first images. Optionally, said position-marking probe is selected from a group consisting of a therapeutic probe, a thermal sensor probe, a heating probe, and an echogenic probe easily visible under said imaging modality.

In an exemplary embodiment of the invention, said position-marking probe is a cryoprobe. Optionally, said cryoprobe is fixed in position within said body portion by being cooled to freezing temperature, thereby causing adherence between said cryoprobe and body tissues.

In an exemplary embodiment of the invention, the method comprises issuing a warning if (f) fails to yield a satisfactory predicted outcome. Optionally, the method comprises generating a suggested therapy plan, including at least one change by a computerized planner having a better predicted outcome that shown by said calculating. Optionally, the method comprises repeating said (e) and (f) after applying of said suggested therapy plan.

In an exemplary embodiment of the invention, (e) comprises using said second image to redefine a treatment goal.

In an exemplary embodiment of the invention, the method comprises using said second image to redefine a treatment goal due to shifting of target tissue by probes.

In an exemplary embodiment of the invention, the method comprises generating, by a computerized planner, a suggested retraction of a probe.

There is provided in accordance with an exemplary embodiment of the invention, a method for simulation and prediction of surgical results, comprising:

(a) establishing a three-dimensional model of a segment of a body of a patient;

(b) establishing within said model planned positions and temperatures of therapeutic devices;

(c) calculating, for at least a portion of said model, a temperature distribution expected to result from use of said therapeutic devices at said planned positions and temperatures;

(d) calculating probabilities of tissue survival outcomes at said calculated temperatures;

(e) displaying said calculated probabilities.

In an exemplary embodiment of the invention, establishing a three-dimensional model of a segment of a patient's body comprises algorithmic analysis of images. Optionally, establishing a three-dimensional model of a segment of a patient's body comprises presenting to a user at least one image of said body segment produced by an imaging modality, and receiving input from said user, said input serving to identify an anatomical feature present in said segment of said body and recognized by said user in said image. Optionally, the method comprises providing to said user a graphical feature marker image expected to resemble a selected anatomical feature, for use in marking said anatomical feature on said image. Optionally, said presented graphical feature marker is selected from a database of graphical feature markers.

Optionally, said graphical feature marker is selected from said database of graphical feature markers according to similarity between said body of said patient and another patient body from whom said selected graphical feature marker image derives.

Optionally, the method comprises accepting said input from a user with respect to a first image, reproducing said user input from said first image on a second image, and enabling said user to identifying an anatomical feature present in said second image by modifying said reproduced input with respect to said second image.

In an exemplary embodiment of the invention, the method comprises interpolating between a position of a first marker on a first image and a position of a second marker on a second image to calculate a proposed position of a third marker on a third image.

In an exemplary embodiment of the invention, said therapeutic devices comprise a heating device serving to protect first tissues during cryoablation of second tissues.

In an exemplary embodiment of the invention, said heating device comprises one of a group consisting of a rectal warmer, a urethral warmer, and a heating needle positioned near a neurovascular bundle.

In an exemplary embodiment of the invention, establishing a three-dimensional model of a segment of a patient's body comprises assigning to at least one tissue represented in an image a tissue-preservation-desirability score, said score being selected from a graduated scale of scores varying, over a plurality of gradations, between desirable to be destroyed and desirable to be preserved.

In an exemplary embodiment of the invention, the method comprises presenting said image to a user, and receiving input from said user specifying said tissue-preservation-desirability score.

In an exemplary embodiment of the invention, the method comprises calculating a tissue-preservation-desirability score as a function of image intensity of pixels of said image.

In an exemplary embodiment of the invention, displaying said calculated probabilities of tissue survival further comprises displaying graphical elements correlated with tissue-preservation-desirability scores.

In an exemplary embodiment of the invention, said stop of establishing within said model planned positions and temperatures of therapeutic devices comprises receiving cryoprobe position designations from a user.

In an exemplary embodiment of the invention, said establishing within said model planned positions and temperatures of therapeutic devices comprises receiving cryoprobe operating parameter designations from a user.

In an exemplary embodiment of the invention, said establishing within said model planned positions and temperatures of therapeutic devices comprises receiving cryoprobe position designations from an algorithmically based recommender system.

In an exemplary embodiment of the invention, said establishing within said model planned positions and temperatures of therapeutic devices comprises imaging a body segment having inserted cryoprobes and establishing within said model cryoprobe positions corresponding to real-time positions of said inserted cryoprobes as shown by said imaging. Optionally, said establishing cryoprobe positions within said model based on said imaging comprises receiving input from a user.

In an exemplary embodiment of the invention, said establishing cryoprobe positions within said model based on said imaging comprises algorithmic analysis of an image produced by said imaging.

There is provided in accordance with an exemplary embodiment of the invention, a method for display of calculated expected outputs of an ablation procedure, comprising

(a) calculating a sequence of temperature maps of a portion of a body over time, said calculation being based on a pre-defined set of cryoprobe position coordinates and a schedule of operating parameters of said cryoprobes over time;

(b) displaying information derived from said maps sequentially to a user.

In an exemplary embodiment of the invention, said displaying comprises displaying an image sequence of kill probability.

In an exemplary embodiment of the invention, said displaying comprises displaying an image sequence of ice-ball boundaries.

In an exemplary embodiment of the invention, timing of displays of said sequence of said information displays is controllable by a user.

In an exemplary embodiment of the invention, said displayed maps display temperature differences as differences of image pixel color intensities.

In an exemplary embodiment of the invention, the method comprises calculating probabilities of tissue destruction as a function of both degree of cooling and time of cooling. Optionally, the method comprises displaying differences among said calculated probabilities as differences in image pixel color intensities.

Optionally, the method comprises calculating probabilities of tissue destruction as a function of degree of cooling, of time of cooling, and of tissue type.

Optionally, at lest one of said displayed maps represents temperatures at an intersection of a two-dimensional plane and a three-dimensional model of at least a portion of a body. Optionally, selection of position, size, and orientation of said displayed two-dimensional plane is at least partially controlled by a user.

In an exemplary embodiment of the invention, the method comprises display of minimal expected temperatures.

In an exemplary embodiment of the invention, the method comprises display of extreme expected temperatures.

In an exemplary embodiment of the invention, the method comprises display of expected percentage of tissue destruction at a selected treatment time at a user-selected locus.

In an exemplary embodiment of the invention, the method comprises display wherein sub-pixel light intensities are calculated as functions of expected percentage of tissue destruction and of scores of desirability of tissue destruction. Optionally, sub-pixel light intensities are calculated as a function of a correlation between expected percentage of tissue destruction and scores of desirability of tissue destruction.

In an exemplary embodiment of the invention, the method comprises user-commanded display of pixel color values calculated as function of a correlation between expected percentage of tissue destruction and scores of desirability of tissue destruction, for locations on a user-selected plane.

In an exemplary embodiment of the invention, the method comprises displaying a graph of a tissue condition over time for a specific tissue location.

There is provided in accordance with an exemplary embodiment of the invention, a cryoprobe having a shaft comprising markings visible under an imaging modality while said cryoprobe is inserted in a patient and an operating tip of said cryoprobe is encased in an ice-ball generated by operation of said probe, said markings indicating distances of said markings from said tip.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified block diagram of a planning system for planning a cryoablation procedure, according to methods of prior art;

FIGS. 2a and 2b are a flow chart showing a method for automatically generating a recommendation relating to a cryoablation procedure, according to methods of prior art;

FIG. 3a is a is a simplified block diagram of a system for facilitating a cryosurgery ablation procedure, according to methods of prior art;

FIG. 3b is a schematic diagram of mechanisms for control of cryosurgical tools by a surgical facilitation system, according to methods of prior art;

FIG. 4 is a simplified schematic of a system for planning and performing cryoablation, according to an embodiment of the present invention;

FIG. 5 is a simplified flowchart of a method for planning and managing a surgical intervention, according to an embodiment of the present invention;

FIG. 6a is a raw ultrasound image of a prostate its vicinity; and

FIG. 6b is a sample user input screen including the image of FIG. 6a after annotation by a user, according to an embodiment of the present invention;

FIG. 6c is a sample user input screen of FIG. 6b, further showing predicted isotherms and recommended probe locations, according to an embodiment of the present invention; and

FIGS. 7a, 7b, and 7c are simplified schematics comparing differences in ablation volume contours produced by synchronized cooling of probes, anti-synchronized cooling of probes, and cooling of a probe while heating a neighboring probe respectively, according to an embodiment of the present invention.

### DESCRIPTION OF THE PREFERRED EMBODIMENTS

The present invention relates to devices and methods for planning and supervising minimally invasive surgery. Specifically, the present invention can be used to enhance various imaging modalities used before and during cryosurgery, to enhance and facilitate user-input characterization of body tissues based on images provided by imaging modalities, to output predictions based on simulated and actual surgical situations in a form well suited to guiding a surgeon in decision-making processes, and to enhance controlled contouring of a cryoablation volume produced by a plurality of cryoprobes.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is further to be understood that some aspects of the present invention are presented hereinbelow in the context of discussions of an exemplary utilization, namely that of cryoablative surgery and contexts for planning and executing surgery by cryocooling. It is to be understood that the context of the examples provided is exemplary only, and not to be regarded as limiting. With the exception of inventive aspects specifically related to cooling and effects of cooling, the invention herein described is not limited to the contexts of cryosurgery, and indeed are expected to be useful in a broad variety of clinical contexts not limited to cryosurgery. In this sense, references below to “cryoprobes” are to be understood as being exemplary and not limiting: references to “cryoprobes” may thus be understood to refer to therapeutic probes in general. That is, the term “cryoprobe” may be read as referring to any probe-like device used to penetrate into a body of a patient for therapeutic or diagnostic or investigative purposes).

As used herein the terms “about” and “approximately” refer to ±20%.

In discussion of the various figures described hereinbelow, like numbers refer to like parts. The drawings are generally not to scale

For clarity, non-essential elements are omitted from some of the drawings.

To enhance clarity of the following descriptions, the following terms and phrases will first be defined:

The phrases “heat exchanger” and “heat-exchanging configuration” are used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when expanded through a Joule-Thomson heat exchanger. As is well known in the art, when gasses such as argon, nitrogen, air, krypton, CO₂, CF₄, and xenon, and various other gasses, at room temperature or colder, pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which, when passed at room temperature or warmer through a Joule-Thomson heat exchanger, have the property of becoming hotter. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating. A Joule-Thomson heater/cooler is thus a “Joule-Thomson heat exchanger” as defined above.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperature by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

The term “registration” as applied to images, physical systems and three-dimensional models in virtual space refers to processes of ascertaining relationships between positions, orientations, and scale of said images, physical systems and three-dimensional models so as to enable to relate distances and dimensions in one of said elements to distances and dimensions in others of said elements

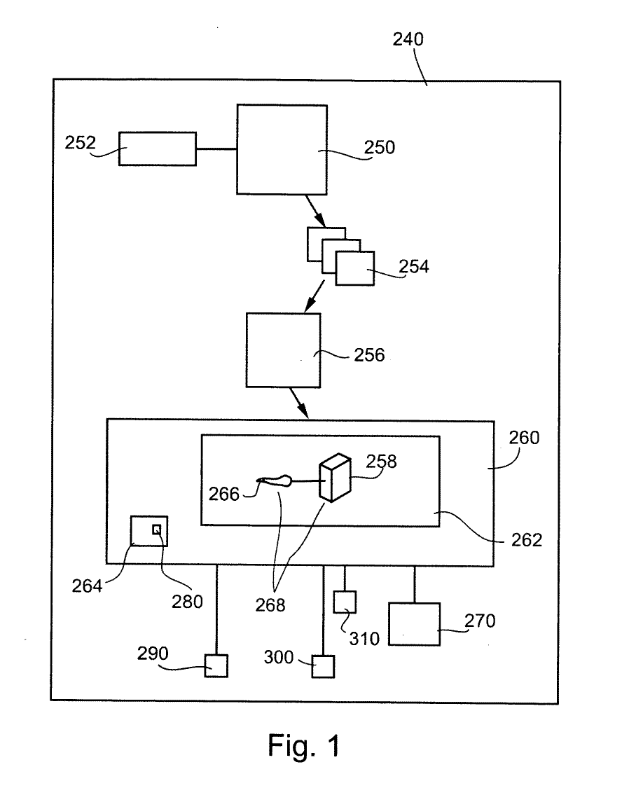

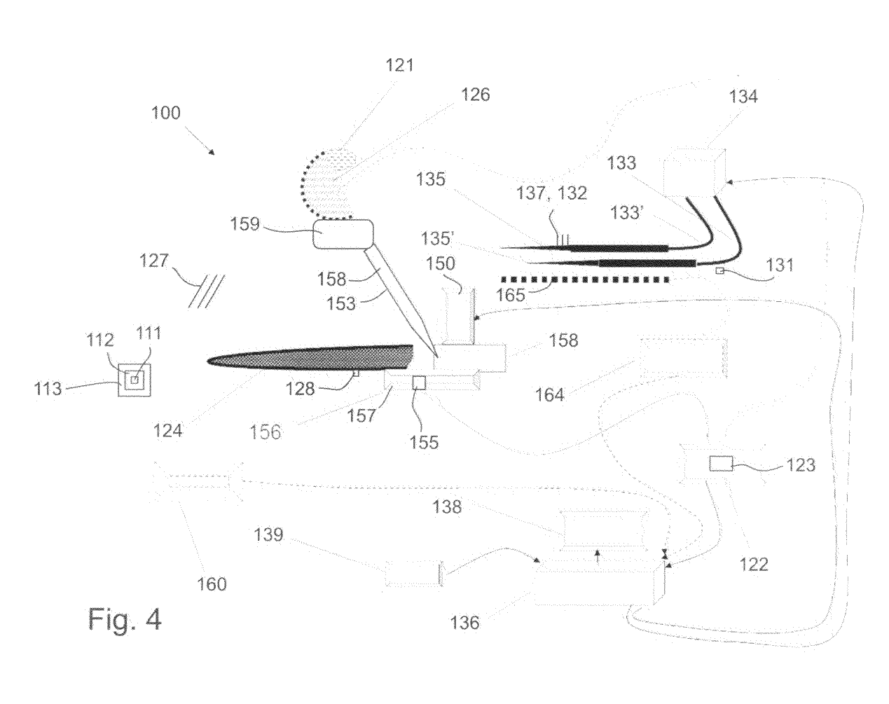

For purposes of better understanding the present invention, as illustrated in FIGS. 4-7 of the drawings, reference is first made to the construction and operation of a conventional (i.e., prior art) surgical planning system, as illustrated in FIGS. 1-3.

Reference is now made to FIG. 1, which is a simplified block diagram of a planning system for planning a cryoablation procedure, according to methods of prior art.

In FIG. 1, a planning system 240 for planning a cryoablation procedure comprises a first imaging modality 250 which serves for creating digitized preparatory images 254 of a cryoablation intervention site. First imaging modality 250 will typically be a magnetic resonance imaging system (MRI), an ultrasound imaging system, a computerized tomography imaging system (CT), a combination of these systems, or a similar system able to produce images of the internal tissues and structures of the body of a patient. First imaging modality 250 is for producing digitized images of a cryoablation intervention site, which site includes body tissues whose cryoablation is desired (referred to herein as “target” tissue), which may be a tumor or other structure, and body tissues and structures in the immediate neighborhood of the target tissues, which constitute the target tissue's physical environment.

Some types of equipment useable as first imaging modality 250, a CT system for example, typically produce a digitized image in a computer-readable format. If equipment used as first imaging modality 250 does not intrinsically produce digitized output, as might be the case for conventional x-ray imaging, then an optional digitizer 252 may be used to digitize non-digital images, to produce digitized preparatory images 254 of the site.

Digitized images 254 produced by first imaging modality 250 and optional digitizer 252 are passed to a three-dimensional modeler 256 for creating a three-dimensional model 258 of the intervention site. Techniques for creating a three dimensional model based on a set of two dimensional images are well known in the art. In the case of CT imaging, creation of a three dimensional model is typically an intrinsic part of the imaging process. PROVISION (http://www.algotec.com/web/products/provision.htm), from Algotec Inc., a division of Eastman Kodak Inc. based in Raanana, Israel, is an example of software designed to make a 2-D to 3-D conversion for images generated by CT scans. To accomplish the same purpose starting from ultrasound imaging, SONOReal™ software from BIOMEDICOM (http://www.biomedicom.com/) may be used.

Three dimensional model 258 is preferably expressible in a three dimensional Cartesian coordinate system.

Three dimensional model 258 is useable by a simulator 260 for simulating a cryosurgical intervention. Simulator 260 comprises a displayer 262 for displaying views of model 258, and an interface 264 useable by an operator for specifying loci for insertion of simulated cryoprobes 266 and operational parameters for operation of simulated cryoprobes 266 for cryoablating tissues. Thus, an operator (i.e., a user) can use simulator 260 to simulate a cryoablation intervention, by using interface 264 to command particular views of model 258, and by specifying both where to insert simulated cryoprobes 266 into an organ imaged by model 258, and how to operate cryoprobes 266. Typically, an operator may specify positions for a plurality of simulated cryoprobes 266, and further specify operating temperatures and durations of cooling for cryoprobes 266. Display 262 is then useable for displaying in a common virtual space an integrated image 268 comprising a display of three dimensional model 258 and a virtual display of simulated cryoprobes 266 inserted at said operator-specified loci.

Planning system 240 optionally comprises a memory 270, such as a computer disk, for storing operator-specified loci for insertion of cryoprobes and operator-specified parameters for operation simulated cryoprobes 266.

Interface 264 comprises a highlighter 280 for highlighting, under control of an operator, selected regions within three dimensional model 258. Operator-highlighted selected regions of model 258 are then optionally displayed as part of an integrated image 268.

In particular, highlighter 280 is useable by an operator for identifying tissues to be cryoablated. Preferably, interface 264 permits an operator to highlight selected regions of three dimensional model 258 so as to specify therein tissues to be cryoablated, or alternatively interface 264 permits an operator to highlight selected regions of digitized preparatory images 254, specifying therein tissues to be cryoablated. In the latter case, three-dimensional modeler 256 is then useable to translate regions highlighted on digitized preparatory images 254 into equivalent regions of three dimensional model 258. In both cases, tissues highlighted and selected to be cryoablated can be displayed by displayer 262 as part of integrated image 268, and can be recorded by memory 270 for future display or other uses.

Similarly, highlighter 280 is useable by an operator for identifying tissues to be protected from damage during cryoablation. Typically, important functional organs not themselves involved in pathology may be in close proximity to tumors or other structures whose destruction is desired. For example, in the case of cryoablation in a prostate, nerve bundles, the urethra, and the rectum may be in close proximity to tissues whose cryoablation is desired. Thus, highlighter 280 is useable by an operator to identify (i.e., to specify the location of) such tissues and to mark them as requiring protection from damage during cryoablation.

Preferably, interface 264 permits an operator to highlight selected regions of three dimensional model 258 so as to specify therein tissues to be protected from damage during cryoablation. Alternatively, interface 264 permits an operator to highlight selected regions of digitized preparatory images 254, specifying therein tissues to be protected during cryoablation. In the latter case, three-dimensional modeler 256 is then useable to translate regions highlighted on digitized preparatory images 254 into equivalent regions of three dimensional model 258. In both cases, tissues highlighted and selected to be protected from damage during cryoablation can be displayed by displayer 262 as part of integrated image 268, and can be recorded by memory 270 for future display or other uses.

Planning system 240 further optionally comprises a predictor 290, an evaluator 300, and a recommender 310.

Predictor 290 serves for predicting the effect on tissues of a patient, if a planned operation of cryoprobes 266 at the operator-specified loci is actually carried out according to the operator-specified operational parameters. Predictions generated by predictor 290 may optionally be displayed by displayer 262 as part of integrated image 268, in the common virtual space of image 268.

In a preferred embodiment, predictions of predictor 290 are based on several sources. The laws of physics, as pertaining to transfer of heat, provide one predictive source. Methods of calculation well known in the art may be used to calculate, with respect to any selected region within three dimensional model 258, a predicted temperature, given known locations of cryoprobes 266 which are sources of cooling in proximity to such a region, known temperatures and cooling capacities of cryoprobes 266, and a duration of time during which cryoprobes 266 are active in cooling. Thus, a mathematical model based on known physical laws allows to calculate a predicted temperature for any selected region within model 258 under operator-specified conditions.

Experimentation and empirical observation in some cases indicate a need for modifications of a simple mathematical model based on physical laws concerning the transfer of heat, as would be the case, for example, in a tissue wherein cooling processes were modified by a high rate of blood flow. However, methods for adapting such a model to such conditions are also well known in the art. Such methods take into account heat dissipation in flowing systems, affected by the flow.

An additional basis for predictions of predictor 290 is that of clinical observation over time. Table 1 provides an example of a predictive basis derived from clinical observation, relating to medium-term and long-term effects of cryoablation procedures in a prostate. The example provided in Table 1 relates to treatment of BPH by cryoablation under a standardized set of cryoprobe operating parameters.

TABLE 1
Predicted long-term effects of cryoablation

| Distance between | 3 week volume | 3 months volume |
|---|---|---|
| probes (mm) | consumption (%) | consumption (%) |
| 10 | 70 | 100 |
| 15 | 55 | 85 |
| 20 | 40 | 70 |
| 25 | 30 | 50 |

As may be seen from Table 1, clinical observation leads to the conclusion that reduction in the volume of a prostate following cryoablation is a gradual process which continues progressively for a number of weeks following a cryoablation procedure. The clinically derived information of Table 1, and similar clinically derived information, can also serve as a basis for predictions generated by predictor 290, and displayed by displayer 262 as part of integrated image 268 in the common virtual space of image 268.

Evaluator 300 is useable to compare results predicted by predictor 290 to goals of a surgical intervention as expressed by an operator. In particular, evaluator 300 can be used to compare intervention results predicted by predictor 290 under a given intervention plan specified by an operator, with that operator's specification of tissues to be cryoablated. Thus, an operator may use interface 264 to specify tissues to be cryoablated, plan an intervention by using interface 264 to specify loci for insertion of cryoprobes 266 and to specify a mode of operation of cryoprobes 266, and then utilize predictor 290 and evaluator 300 to predict whether, under his specified intervention plan, his/her goal will be realized and all tissues desired to be cryoablated will in fact be destroyed. Similarly, an operator may utilize predictor 290 and evaluator 300 to predict whether, under his/her specified intervention plan, tissues which he specified as requiring protection from damage during cryoablation will in fact be endangered by his planned intervention.

Recommender 310 may use predictive capabilities of predictor 290 and evaluator 300, or empirically based summaries of experimental and clinical data, or both, to produce recommendations for cryoablation treatment.

As discussed above, predictor 290 and evaluator 300 can be used to determine, for a given placement of a given number of cryoprobes and for a given set of operating parameters, whether a planned cryoablation procedure can be expected to be successful, success being defined as destruction of tissues specified as needing to be destroyed, with no damage or minimal damage to tissues specified as needing to be protected during cryoablation. Based on this capability, recommender 310 can utilize a variety of calculation techniques well known in the art to evaluate a plurality of competing cryoablation intervention strategies and to express a preference for that strategy which is most successful according to these criteria.

In particular, recommender 310 may consider several intervention strategies proposed by an operator, and recommend the most successful among them. Alternatively, an operator might specify a partial set of operating parameters, and recommender 310 might then vary (progressively or randomly) additional operating parameters to find a ‘best fit’ solution. For example, an operator might specify tissues to be destroyed, tissues to be protected, and a two-dimensional array of cryoprobes such as, for example, the two dimensional placement array of cryoprobes determined by the use of guiding element 115 having a net of apertures 120 shown in FIG. 8 hereinabove. Recommender 310 could then test a multitude of options for displacements of a set of cryoprobes in a third (depth) dimension to determine the shallowest and deepest penetration desirable for each cryoprobe. Recommender 310 could further be used to calculate a temperature and duration of freezing appropriate for each cryoprobe individually, or for all deployed cryoprobes controlled in unison, in a manner designed to destroy all tissues specified to be destroyed, while maximizing protection of tissues specified to be protected.

Recommendation activity of recommender 310 may also be based on empirical data such as experimental results or clinical results. Table 2 provides an example of a basis for making recommendations derived from clinical observation.

TABLE 2
Recommended number of cryoprobes to treat BPH

| American | | | |
|---|---|---|---|
| Urologists | Number of | | |
| Association | cross-sections | | |
| Questionnaire | with stricture of | Prostate | Number |
| Score | the Urethra | Volume | of probes |
| 0-7 | 1-3 | 25 | 2 |
| 0-7 | 1-3 | 40 | 2 |
| 0-7 | 2-5 | 40 | 2 |
| 0-7 | 1-3 | 50 | 2-3 |
| 0-7 | 2-5 | 50 | 2-3 |
| 0-7 | 1-3 | 60 | 2-3 |
| 0-7 | 2-5 | 60 | 3 |
| 0-7 | 2-5 | 100 | 4 |
| 8-19 | 1-3 | 40 | 2-3 |
| 8-19 | 2-5 | 40 | 2-3 |
| 8-19 | 1-3 | 50 | 2 |
| 8-19 | 2-5 | 50 | 2-3 |
| 8-19 | 1-3 | 60 | 3 |
| 8-19 | 2-5 | 60 | 3-4 |
| 8-19 | 2-5 | 100 | 4 |
| 20-35 | 1-3 | 40 | 3 |
| 20-35 | 2-5 | 40 | 3 |
| 20-35 | 1-3 | 50 | 4 |
| 20-35 | 2-5 | 50 | |
| 20-35 | 1-3 | 60 | 4 |
| 20-35 | 2-5 | 60 | 5 |
| 20-35 | 2-5 | 100 | 6 |

Table 2 relates to the treatment of BPH by cryoablation. Table 2 is essentially a table of expert opinion, wherein three criterions for describing the symptomatic state of a patient are related, by experts, to a recommendation for treatment. Table 2 was in fact compiled by a group of experts in the practice of cryoablation utilizing a particular tool, specifically a tool similar to that described in FIG. 8 hereinabove, yet a similar table may be constructed by other experts and for other tools. Moreover, feedback from the collective clinical experience of a population of users of a particular tool may be collected over time, for example by a company marketing such a tool or by an independent research establishment, and such collected information may be fed back into recommender 310 to build a progressively better informed and increasingly useful and reliable recommendation system.

The first column of Table 2, the AUA score, is the score of a questionnaire in use by the American Urological Association which may be found in Tanagho E. A., and McAninich J. W., Smith's General Urology, published by McGraw-Hill, Chapter 23. The AUA score is an estimate of severity of symptoms as subjectively reported by a patient, and relates to such urinary problems as incomplete emptying of the bladder, frequency of urination, intermittency, urgency, weak stream, straining, nocturia, and the patient's perceived quality of life as it relates to his urinary problems.

The second and third columns of Table 2 relate to diagnostic criteria discernable from three-dimensional model 258 or from digitized preparatory images 254 from which model 258 derives. The second column is a measure of the length of that portion of the urethra observed to be constricted by pressure from a patient's prostate. The third column is a measure of the volume of that patient's prostate. Table 2 constitutes a basis for recommending an aspect of a cryoablation treatment for BPH, specifically for recommending, in column four, an appropriate number of cryoprobes to be used in treating a specific patient, based on three quantitative evaluations of his condition constituted by the columns one, two and three of Table 2.

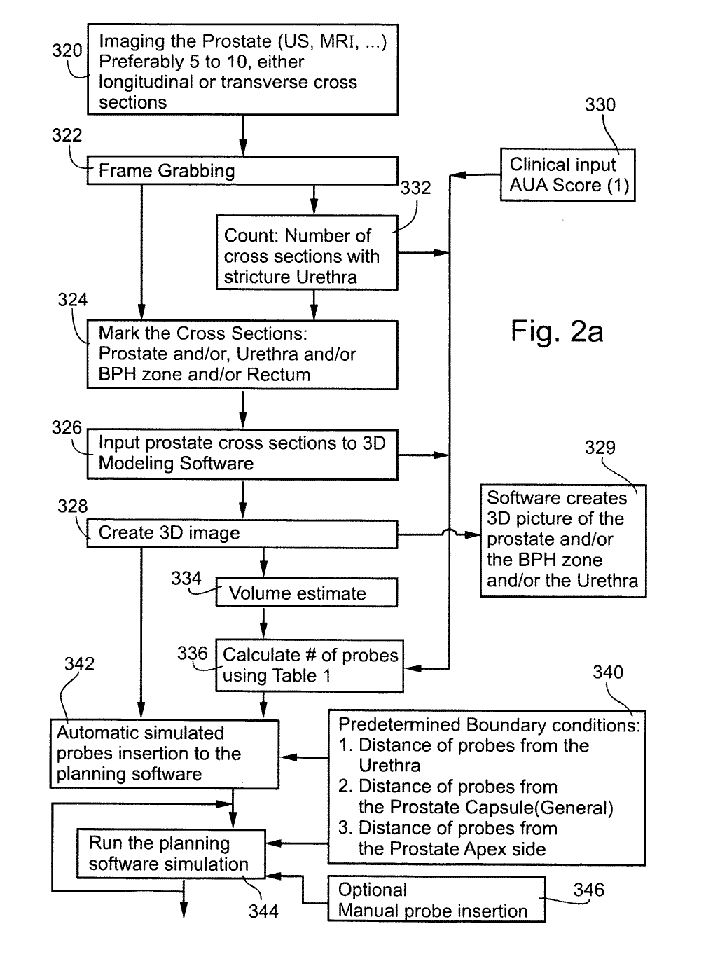

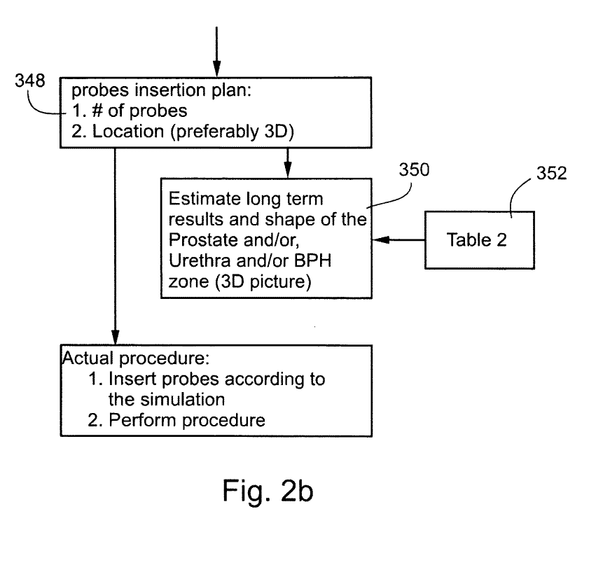

Reference is now made to FIGS. 2a and 2b, presenting a flow chart showing a method for automatically generating a recommendation relating to a cryoablation procedure, utilizing the information of Table 2, or similar information, according to methods of prior art. In the specific example of FIGS. 2a-2b, the generated recommendation is relevant to cryoablation of tissues of a prostate for treatment of BPH.

At step 320 of FIG. 2a, first imaging modality 250 is used to create preparatory images, which are digitized at step 322 to become digitized preparatory images 254. In the example presented, images 254 are cross sections of a prostate such as those generated by a series of ultrasound scans taken at regularly intervals of progressive penetration into the body of a patient, as might be produced by the ultrasound equipment described with reference to FIGS. 8-10 hereinabove.

At optional step 324, an operator marks or otherwise indicates, with reference to images 254, locations of tissues to be cryoablated or to be protected, as explained hereinabove. At step 326 images 254 are input to three-dimensional modeler 256, which creates three-dimensional model 258 of the intervention site at step 328. Model 258, along with any operator-highlighted and classified regions of model 258, are displayed at step 329.

In a parallel process, raw materials for a recommendation are gathered. At step 330 clinical input in the form of an AUA score from a questionnaire of a patient's symptoms is input. At step 332 a count is made of the number of preparatory images 254 (cross-sections) of the urethra which show constriction to the urethra caused by pressure from the prostate tissue on the urethra. A count of cross-sections showing constriction is here taken as an indication of the length of a stricture. Determination of which cross-section images show signs of constriction may be made by an operator, or alternatively may be made by automated analysis of images 256, using image interpretation techniques well known in the art. At step 334, information available to three-dimension modeler 256 is used to automatically calculate the volume of the prostate.

At step 336, information assembled at steps 330, 332, and 334 is used in a table-lookup operation to retrieve a recommendation for the appropriate number of probes to be used to treat the imaged specific case of BPH.

At step 340, an operator optionally inputs specific boundary conditions which serve to limit recommendations by the system. Utilizing model 254 created at step 328, operator-specified boundary conditions from step 340, operator-specified identification of locations of specific tissues to be ablated or protected from step 324, and a calculated recommended number of probes from step 336, a recommendation for optimal positioning of a recommended number of probes may be made at step 342. Display of a recommended intervention is made at step 344.

Optionally, operator-specified placement of simulated cryoprobes may modify or replace the recommended intervention, at step 346.

Step 344 is optionally iterative. That is, an operator may repeatedly modify definitions of tissues, boundary conditions, or manual placement of simulated probes, until the operator is satisfied with the simulated results. As a part of step 344, activities of evaluator 300 may be evoked, so as to procure system feedback based on a simulated intervention. Step 344 is repeated so long as desired by an operator, and until the operator is satisfied with the results.

Referring now to FIG. 2b which is a continuation of the flowchart of FIG. 2a, at step 348 a final plan is optionally saved to a computer disk or other memory 270.

In optional step 350, details of the completed intervention plan can be used to estimate and display expected long-term results of the planned intervention, such as an expected future volume and shape of the prostate. Information from Table 2 or an equivalent is utilized for step 350, as indicated at step 352. It is noted that long-term volume of the prostate may also be treated as a boundary condition of an intervention, at step 340.

The example presented in FIGS. 2a and 2b refers specifically to a utilization of planning system 240 for treating a prostate for BPH. Similar utilizations may be contemplated, for treating other organs, or for treating other conditions of a prostate.

In treating BPH, a desired goal is a reduction in prostate volume so as to relieve pressure on the urethra of a patient, because pressure on the urethra from an enlarged prostate interferes with the process of urination. In treating BPH there is no need to destroy all of a selected volume, but rather simply to destroy some desired percentage of that volume.

In treating, for example, a prostate tumor suspected of malignancy, goals of the intervention are quite different. To avoid dangerous proliferation of malignant cells, it is desirable to ablate a defined volume in its entirety. In such a context, when it is necessity to destroy all tissues within a selected volume, the functionality of evaluator 300 of planning system 240 is particularly useful.

Evaluator 300 is able to calculate, for each arbitrarily selected small volume of model 258, the cumulative cooling effect of all cryoprobes in proximity to said selected small volume. Consequently evaluator 300 is able to make at least a theoretical determination of whether, for a given deployment of cryoprobes utilized under a given set of operating parameters, total destruction of malignant tissues within a selected volume is to be expected.

Planning system 240 can be used effectively to plan a dense arrays of cryoprobes. For example, a user might specify a particular density of an array of probes, then use evaluator 300 to evaluate a range of possible temperature and duration parameters to find an amount and duration of cooling which ensures that the specified array will indeed create a nearly-uniform cold field sufficient to destroy all target tissues. Alternatively, a user might specify a desired degree of cooling and use planning system 240 to recommend a required density of the cryoprobe array.

Thus, evaluator 300 and recommender 310 can be used to calculate placement and operational parameters of cryoprobes in a manner which guarantees a nearly-uniform cold field within a selected volume. If cryoprobes 266 are sufficiently small and placed sufficiently close together, cooling effects from a plurality of probes will influence each selected small volume within a target volume, and an amount of required cooling can be calculated which will ensure that all of the target volume is cooled down to a temperature ensuring total destruction of the target volume.

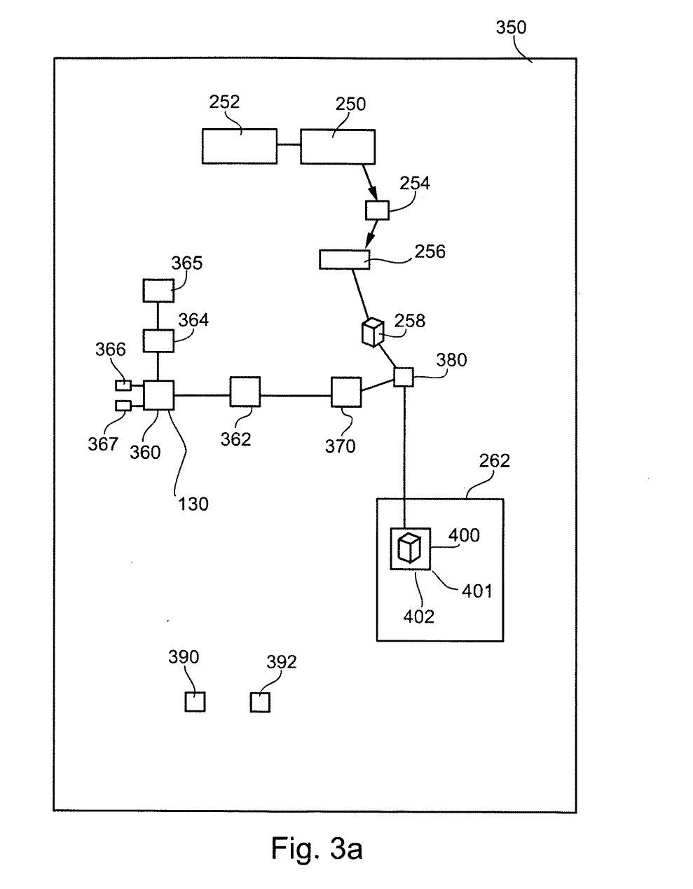

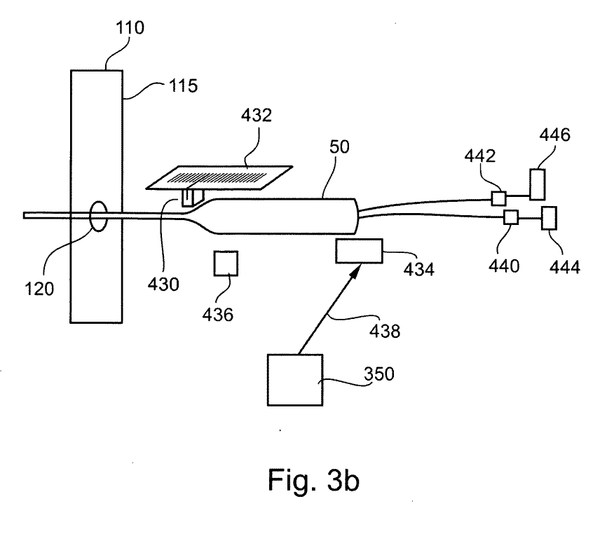

Reference is now made to FIG. 3a, which is a simplified block diagram of a surgical facilitation system for facilitating a cryosurgery ablation procedure, according to methods of prior art.

In a preferred embodiment, a surgical facilitation system 350 comprises a first imaging modality 250 and optional digitizer 252, for creating digitized preparatory images 254 of an intervention site, a first three-dimensional modeler 256 for creating a first three-dimensional model 258 of the intervention site based on digitized preparatory images 254, a second imaging modality 360 with optional second digitizer 362 for creating a digitized real-time image 370 of at least a portion of the intervention site during a cryosurgery procedure, and an images integrator 380 for integrating information from three-dimensional model 258 of the site and from real-time image 370 of the site in a common coordinate system 390, thereby producing an integrated image 400 displayable by a display 260. Integrated image 400 may be a two dimensional image 401 created by abstracting information from a relevant plane of first three dimensional model 258 for combining with a real-time image 370 representing a view of that plane of that portion of the site in real-time. Alternatively, a set of real-time images 370 may be used by a second three dimensional modeler 375 to create a second three dimensional model 402, enabling images integrator 380 to express first three dimensional model 258 and second three dimensional model 402 in common coordinate system 390, preferably a Cartesian coordinate system, thereby combining both images into integrated image 400.

Various strategies may be used to facilitate combining of model 258 (based on preparatory images 254) with real-time images 370 (or model 402 based thereupon) by images integrator 380. Processes of scaling of images to a same scale, and of projection of a ‘slice’ of a three dimensional image to a chosen plane, are all well known in the art. Basic techniques for feature analysis of images are also well known, and can deal with problems of fine alignment of images from two sources, once common features or common directions have been identified in both images. Techniques useful for facilitating aligning of both images by images integrator 380 include: (a) identification of common features in both images by an operator, for example by identifying landmark features such as points of entrance of a urethra into, and points of exit of a urethra from, a prostate, (b) identification of constant basic directions, such as by assuring that a patient is in a similar position (e.g., on his back) during both preparatory imaging and real-time imaging, (c) operator-guided matching, through use of interface 264, of a first set of images, (d) use of proprioceptive tools for imaging, that is, tools capable of reporting, either mechanically or electronically using an electronic sensor 364 and digital reporting mechanism 365, their own positions and movements, and (e) using a same body of imaging equipment to effect both preparatory imaging, producing preparatory images 254, and real-time imaging during a cryosurgery procedure, producing real-time images 370. For example, using ultrasound probe 130 of FIG. 3a both for preparatory imaging and for real-time imaging, and assuring that the patient is in a standard position during both imaging procedures, greatly facilitates the task of images integrator 380. Equipping ultrasound probe 130 with stabilizer 366 and controlling its movements with stepper motor 367, as shown in FIG. 3a, yet further simplifies the task of images integrator 380.

It will be appreciated that the described system can benefit from position tracking of various components thereof so as to assist either in modeling and/or in actually controlling a cryoablation procedure. Position tracking systems per se are well known in the art and may use any one of a plurality of approaches for the determination of position in a two- or three-dimensional space as is defined by a system-of-coordinates in two, three and up to six degrees-of-freedom. Some position tracking systems employ movable physical connections and appropriate movement monitoring devices (e.g., potentiometers) to keep track of positional changes. Thus, such systems, once zeroed, keep track of position changes to thereby determine actual positions at all times. One example for such a position tracking system is an articulated arm. Other position tracking systems can be attached directly to an object in order to monitor its position in space. An example of such a position tracking system is an assortment of three triaxially (e.g., co-orthogonally) oriented accelerometers which may be used to monitor the positional changes of the object with respect to a space. A pair of such assortments can be used to determine the position of the object in six-degrees of freedom.

Other position tracking systems re-determine a position irrespective of previous positions, to keep track of positional changes. Such systems typically employ an array of receivers/transmitters which are spread in known positions in a three-dimensional space and transmitter(s)/receiver(s), respectively, which are in physical connection with the object whose position being monitored. Time based triangulation and/or phase shift triangulation are used in such cases to periodically determine the position of the monitored object. Examples of such a position tracking systems employed in a variety of contexts using acoustic (e.g., ultrasound) electromagnetic radiation (e.g., infrared, radio frequency) or magnetic field and optical decoding are disclosed in, for example, U.S. Pat. Nos. 5,412,619; 6,083,170; 6,063,022; 5,954,665; 5,840,025; 5,718,241; 5,713,946; 5,694,945; 5,568,809; 5,546,951; 5,480,422 and 5,391,199, which are incorporated by reference as if fully set forth herein.

Position tracking of any of the imaging modalities described herein and/or other system components, such as the cryoprobes themselves, and/or the patient, can be employed to facilitate implementation of the present invention.

In a preferred embodiment, surgical facilitation system 350 further comprises all functional units of planning system 240 as described hereinabove. That is, facilitation system 350 optionally comprises simulator 260 having user interface 264 with highlighter 280, each having parts, functions and capabilities as ascribed to them hereinabove with reference to FIG. 1 and elsewhere. In particular, system 350 includes the above-described interface useable by an operator to specify placements and operational parameters of simulated cryoprobes 266, and to specify tissues to be cryoablated or to be protected during cryoablation.

Similarly, facilitation system 350 further optionally comprises memory 270, predictor 290, evaluator 300, and recommender 310, each having parts, functions and capabilities as ascribed to them hereinabove with reference to FIG. 1 and elsewhere.

Thus, in a preferred embodiment of the described system, facilitation system 350 is able to undertake all activities described hereinabove with respect to planning system 240. In addition, facilitation system 350 is able to provide a variety of additional services in displaying and evaluating at least one real-time image 370, and is further able to compare real-time images 370 to three dimensional model 258, and also to compare information from real-time images 370 to stored information such as that identifying operator-specified tissues to be cryoablated or to be protected, as is explained more fully hereinbelow.

In a preferred embodiment, either first imaging modality 250 and/or second imaging modality 360 may each independently be a magnetic resonance imaging system (MRI), an ultrasound imaging system, a computerized tomography imaging system (CT), some combination of these systems, or some similar system able to produce images of the internal tissues and structures of the body of a patient, yet in the case of second imaging modality 360, ultrasound and MRI imaging are more typically used, as being more conveniently combined with cryosurgery processes.

Facilitation system 350 further comprises a first comparator 390, for comparing first three-dimensional model 248 with real-time image 370, particularly to discern differences between both images. Such differences constitute differences between a status of a planned intervention and a status of an actual intervention in real-time. Tools, such as cryoprobes, tissues, such as a urethra, and ice-balls formed during cryoablation, all figure as elements in three dimensional model 258, and all may be visualized using second imaging modality 360. Thus, their expected positions, sizes, orientations, and behaviors may be compared to their actual real-time positions, sizes, orientations and behaviors during cryoablation, by comparator 390.

Differences thereby revealed, and information concerning such differences, can be of vital importance to an operator in guiding his actions during an intervention, particularly if the operator deviates from a planned intervention without being aware of doing so. A representation of the revealed differences may be displayed by displayer 262 and highlighted for greater visibility. A feedback mechanism 392, for example an auditory feedback mechanism, may be used to draw attention of an operator to serious discrepancies between a planned and an actual intervention.

Similarly, comparator 390 can be used to compare status of objects visible in real-time images 370 with stored information about operator-specified tissues to be cryoablated. Comparator 390 can thus provide information about, and displayer 262 can display, situations in which tissues intended to be cryoablated are in fact not effectively being cryoablated by a procedure. Similarly, comparator 390 can be used to check status of objects visible in real-time images 370, relating them to stored information about operator-specified tissues which are to be protected during cryoablation. In the case of discrepancies between an actual situation and an operator-specified desirable situation, display 262 and feedback mechanism 392 can warn an operator when a procedure seems to be endangering such tissues.

The capabilities of facilitation system 350 may extend yet further, to direct guidance to an operator in the manipulation of cryoablation tools, and even to partial or complete control of such tools during a phase of a cryoablation intervention.

Reference is now made to FIG. 3b, which is a schematic diagram of mechanisms for control of cryosurgical tools by a surgical facilitation system, according to methods of prior art.

A cryosurgical probe 50 is shown passing through an aperture 120 in a guiding element 115 which is realized in this example as a plate 110. Aperture 120 is for limiting sideways movement of probe 50, which is however free to move forward and backwards towards and away from a cryoablation site in a patient. In prior art methods such as that of Schatzberger discussed in the background section hereinabove, such movement is conceived as under sole and exclusive control of an operator who advances and retracts probe 50 manually.

As has been noted above, the simulation, evaluation, and recommendation capacities of planning system 240 and facilitation system 350, based on preparatory images 254 and three dimensional model 258, allow system 350 to calculate a recommended maximum and minimum depth for at which each cryoprobe 50 is to be used for cryoablation. Further, a cryoablation plan manually entered by an operator may also determine a maximum and minimum depth at which each cryoprobe 50 is to be used for cryoablation.

In a simple implementation of mechanical control based on information from planning system 240 or facilitation system 350, planned maximum and minimum depths generated by those systems are communicated to an operator who adjusts a mechanical blocking element 430 according to a graduated distance scale 432, in a manner which limits forward or backward movement of probe 50 so as to prevent an operator from unintentionally and unknowingly advancing or retracting probe 50 beyond limits of movement planned for probe 50. Such an arrangement guides and aids an operator in use and control of probe 50 for effecting cryoablation according to a plan.

In a somewhat more sophisticated implementation, control signals 438 from system 350 activate a stepper motor 434 to directly control movement of probe 50. Thus, under control of system 350 and according to a planned, simulated, examined and theoretically tested procedure, stepper motor 434 can advance probe 50 to a planned depth for performing cryoablation. System 350 can also send temperature control signals to heating gas valve 440 and cooling gas valve 442, thereby controlling a flow of heating gas from heating gas reservoir 444 and a flow of cooling gas from cooling gas reservoir 446. Thus, under control of an intervention plan and utilizing mechanisms presented herein, system 350 is able to directly control some or all of a cryoablation intervention. Thus, in a typical portion of a cryoablation procedure, stepper 434 advances probe 50 a planned distance, cooling gas valve 442 opens to allow passage of a gas which cools probe 50 to cryoablation temperatures and maintains those temperatures for a planned length of time, then cooling valve 442 closes to halt cooling. Optionally, heating gas valve 440 then opens to allow passage of a gas which heats probe 50 so as to melt tissues in contact with probe 50, thereby restoring to it freedom of motion, whereupon stepper motor 434 can further advance or retract probe 50 to a new cryoablation position, at which new position system 350 can optionally repeat this cryoablation process.

To ensure accuracy, movement of cryoprobe 50 may be monitored by a movement sensor 436. Moreover, all the facilities of system 350 previously described, for comparing real-time positions of objects with planned positions of those objects, can be brought to bear, to monitor this independently controlled cryoablation process.

Attention is now drawn to FIG. 4, which is a simplified schematic of a system 100 for planning and monitoring a probe-based surgical procedure such as a cryoablation, according to an embodiment of the present invention.

System 100 may be used to:
acquire one or more images image of a neighborhood of an lesion to be treated, typically utilizing one or more imaging modalities such as ultrasound, CT, MRI, x-ray, or other;
optionally receive information from a user relating to that image(s), in particular information identifying and localizing organs to be treated, information identifying and localizing organs or structures to be protected from damage (which information is referred to hereafter as the “desired outcomes”);
optionally integrate additional information from imaging modalities such as PET, fMRI and Nuclear Medical imaging (NM) and/or from non-imaging sources such as biopsy results, which information indicates probability of malignancy and/or desirability of tissue destruction in specific locations;
optionally receive from user or from a recorded information source a set of a commands and constraints relating to a cryotherapy operation to be planned and/or executed, (e.g. type and number of cryoprobes to be used, desired temperature profiles, optimization constraints, etc.)
plan a cryoablation procedure based on the received information. Planning may involve estimating outcomes of user-input commands and/or recommending probe placements and/or probe operational parameters such as time and intensity of cooling, pull-back protocols etc.;
optionally simulate (i.e. calculate) expected results of the planned cryoablation procedure, optionally displaying calculated results in a variety of formats, preferably including display of two-dimensional maps or three-dimensional models of the body region being treated, showing temperature isotherms and/or zones of probabilities of tissue destruction, preferably highlighting relationships of similarity or dissimilarity between these predicted outcomes and the desired outcomes;
optionally, use automatic means to insert cryoprobes into a patient, and/or assist a surgeon in inserting cryoprobes, and/or monitor insertion of cryoprobes and provide feedback to a surgeon regarding his insertions as related to the planned insertions;
acquire (from imaging modalities optionally annotated by user) information concerning actual positions of inserted cryoprobes after insertion is completed;
optionally, plan a cryoablation procedure based on actual detected cryoprobe positions;
perform and/or monitor performance of cryoablation procedure as planned while displaying process to user and accepting user override commands, optionally providing feedback and/or controlling procedure based on similarities and differences between planned and actual outcomes and/or between actual outcomes and desired outcomes.

FIG. 4 presents an exemplary embodiment of the present invention designated system 100. In addition to components explicitly listed in the following discussion, it is to be understood that embodiments of system 100 may further include any of the components and features presented above in context of discussions of FIGS. 1-4, and may be operable to perform all functions presented in the foregoing discussion.

System 100 comprises a thermal ablation planning unit 136 having a display 138 and input device 139. Planning unit 136 is preferably a computer such as a PC or a laptop computer. Display 138 is preferably a flat panel graphic display such as LCD, but may be a CRT or plasma display, a stereoscopic display device, or other graphic display. Input device 139 preferably comprises a pointing device such as a mouse and may also comprise a keyboard. Optionally, input device 139 comprises a microphone and voiced recognition software for receiving voice commands and/or for recording voice comments. A plurality of input devices may be used.

An ultrasound control unit 122, connected to an internal ultrasound probe 124 is used for acquiring ultrasonic signals enabling to construct ultrasonic images of the tissue under treatment. For example, in treatment of the prostate ultrasound probe 124 is preferably a rectal probe inserted into the patient's rectal cavity, whereas in treatment of the uterus or its vicinity ultrasound probe 124 is preferably a vaginal probe inserted into the patient's vaginal cavity. Alternatively, ultrasound probe 124 may be an internal probe designed for insertion into any other natural or surgically made body cavity. As taught by Schatzberger, ultrasound probe 124 may be attached or otherwise physically related to a probe insertion guide (template) used for guiding insertion of therapeutic probes into a patient. A fixed physical relationship (or other known positional relationship) between probe and template simplifies registration of images provided by probe 124 with known locations of inserted therapeutic probes.

Optionally, internal ultrasound probe 124 may be connected to a motorized probe positioner 156 which is operable to control movement (e.g. advancement/retraction and/or rotation) of internal ultrasound probe 124 into and within the body cavity. Utilizing probe positioner 156 can facilitate acquiring a plurality of images having know spatial relationships one to another, from which a three-dimensional model of the organ or volume to be treated may be formed, as discussed above. In a currently preferred embodiment positioner 156 is used to capture a series of ultrasound images representing 5 mm steps from one image to the next. Each image is then used as a “slice” of a “multi-slice” 3-D image, useable to construct a three-dimensional model of the target organ and its neighborhood. Such a model and/or other form of 3-D image may be stored, used, and manipulated by planning unit 136 and displayed on display 138, optionally in stereoscopic format. Thus, display 138 can be used to display either a series of two-dimensional views or a three-dimensional view of the surgical target area, which views can be used for target analysis and planning. In a further preferred embodiment, a mechanically or electronically steerable ultrasonic probe having multi-dimensional freedom of motion/viewing-angle may be used.

In place of motorized probe motion mechanism 156, a manual probe motion mechanism 157 may be used, by means of which an operator manually manipulates probe 124. Manual mechanism 157 preferably is either designed to move probe 124 by known distance steps, or else comprises a position sensor operable to report probe position, facilitating registration of individual images in a three-dimensional context, enabling 3-D modeling as discussed above.

An external ultrasound probe 126 may be used together with, or instead of, ultrasound probe 124. In a preferred embodiment external ultrasound probe 126 is an abdominal probe. Ultrasound probe 126 may be used from a fixed position, or may be equipped with a position sensor 121. An example of such a sensor is the electromagnetic location sensor CARTO™ XP EP sold by Biosense Webster (Israel) Ltd, Tirat Carmel, ISRAEL, which may be seen at www.biosensewebster.com. Other similar sensors are well known in the art. Optional location sensor 121 provides information on the position and direction from which image views are taken, thereby providing information regarding the spatial relationships between objects visible in disparate views. Such information enables to register ultrasonic images taken by ultrasound probes 124 and 126 within a common fixed Cartesian coordinate system.

Use of a common coordinate system enables, for example, comparing of actual visible ice-ball location and size to planned ice-ball location and size. A common coordinate system is particularly important in cases where ultrasound probe 126 is moved during monitoring, and in cases where images taken for planning purposes are taken from a different viewing point from that used during surgery, or are made by a different imaging device.

Simultaneous use of both external and internal ultrasound probes, or for that matter simultaneous or coordinated use of two or more separated ultrasound probes viewing a same volume from different directions, and reconstitution of a composite image or three-dimensional model based on information supplied by images from both sources, constitutes, in the field of cryosurgery, a significant advance over methods of prior art. It is in the nature of cryosurgery that frozen tissue is opaque to normal ultrasound, consequently viewing from a single perspective cannot provide full information on the condition of a volume undergoing cryoablation. Thus an ultrasound probe inserted in a rectum, as is standard methodology in prostate cryosurgery, cannot reveal with clarity the state of tissues and the position of frozen tissues on the side of the prostate opposite the rectum.

System 100 overcomes this limitation by providing for use during surgery an ultrasound system comprising first and second ultrasound probes, each probe preferably associated with a position reporter operable to report its position and orientation, an image registration system operable to register information gleaned from operation both first and second probes in a common virtual space, and an image display system operable to display an image of a portion of that common virtual space, which image comprises information gleaned from both first and second probes. Position reporters operable to report positions of the probes may be position sensors 121 and 155, or may be functions of a command mechanism such as probe positioner 156 serving to control automated movement of one or both of the probes, or may even be a user interface used by an operator to manual report fixed or moving positions of probes manually controlled by an operator.

According to a preferred method of use, an operator positions a rectal ultrasound probe 126 in a rectum of a patient, and uses that probe to generate a first ultrasound image of a prostate, positioning an abdominal ultrasound probe 126 on the patient's abdomen directed towards the prostate to generate a second ultrasound image of said prostate, and then either simultaneously or alternatively displays the first and second images, providing the user with simultaneous or near-simultaneous views of the prostate from two different perspectives. An ultrasound timing coordinator 123 may be utilized to coordinate rapid alternation of functioning of probes 124 and 126, so as to provide nearly continuous readings from each probe, yet avoid acoustic signal interference between the probes. If rapid alternation of functioning of probes 124 and 126 is used, controller 122 can of course be programmed to avoid flickering of captured images by prolonging display of captured images when appropriate.

In a further preferred embodiment, system 100 uses image registration software to relate information gleaned from the first and second images to a common coordinate system, and displays a composite image of the prostate, the composite image comprising information gleaned from both first and second images. The described method thus produces images providing more complete information than would be provided by either the first or second images alone. This is particularly important in the case of cryosurgery of a prostate, since during cryoablation the large iceball(s) produced by the ablation process prevent ultrasound viewing of the side of the organ which is distant from the ultrasound probe. It is to be noted, however, that whereas this embodiment has been described in the exemplary context of cryoablation of a prostate, the invention is useful in treatment of various organs other than the prostate, and in clinical contexts other than cryoablation.

To aid in registration of images from both probes in a common coordinate system, in a preferred embodiment one or both of probes 124 and 126 are physically connected, optionally through a motorized or manual probe motion mechanism 156 (for probe 124) and/or 129 (for probe 126), to a common physical reference frame 153. Alternatively, some portion of the described mechanisms may be physically connected (e.g. a frame connected to an external ultrasound and also connected to a patient's bed) while other portions rely on position sensors or commandable servomotors to create a known relationship of components (such as probes 126 and 126) to a patient and to each other, that spatial interrelation system being referred to herein as frame 158. Thus a physically connected portion of frame 158 may be connected to a bed, or to a patient, and physically unconnected portions (e.g. an abdominal ultrasound) may be maintained in a known positional relationship to fixed portions of frame 158 by means of position sensors, stepper motors, measuring scales visible to a surgeon, etc.

One or more probe insertion aids 150 may be connected to reference frame 158 and be used for guiding probes cryoprobes (probes 135 and 135′ are seen in this figure) sensor probes, ultrasound probes and other probes to desired locations within the patient's body. Insertion aid 150 may for example be a cryoprobe insertion template similar to that taught by Schatzberger, as cited in the background section hereinabove.

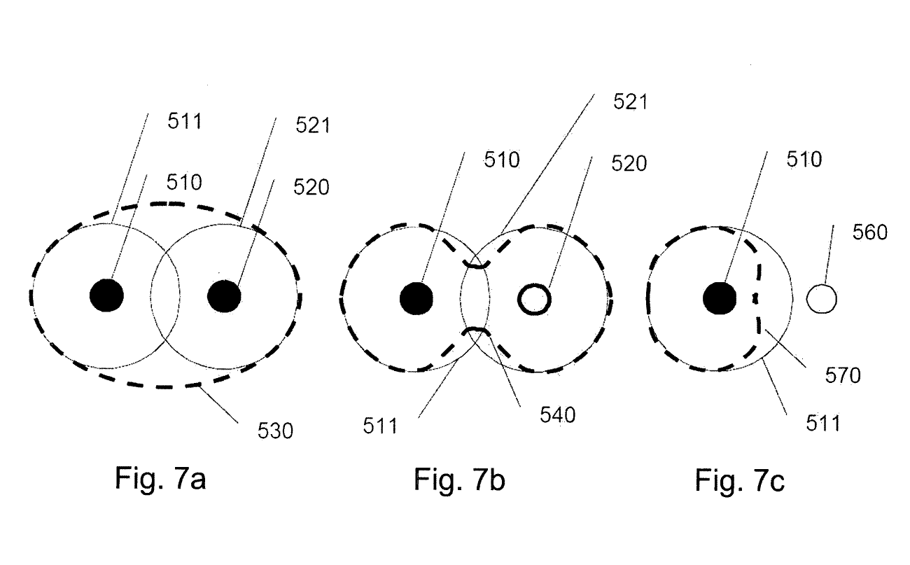

Cryoprobes probes 135 are connected via hoses 133 (two such hoses: 133 and 133′ are seen in this figure) to a cryogen control unit 134 provided to control supply of cryogen to cryoprobes 135, and thereby to control cooling and optionally heating of cryoprobes 135. If cryoprobes 135 are a Joule-Thomson cryoprobes, cryogen control unit 134 will be a controller operable to control supply of high-pressure cooling gas and optionally high-pressure heating gas. Cryogen control unit 134 supplies cryogen through hoses 133 to cryoprobes 135, where it traverses the shaft of cryoprobe 135 and is delivered to operating tips of cryoprobes 135, which tips are cooled by expansion of the cryogen (in the case of a Joule-Thomson cryoprobe) or by evaporation of the cryogen (in the case of an evaporative cryoprobe). Cryogen control unit 134 may be controlled by thermal ablation control unit 136, or alternatively may be manually controlled a user. In a preferred embodiment of system 100 discussed in detail hereinbelow with respect to a pattern of cryoprobe used presented in FIGS. 7a-7c, probes 135 are each operable both to heat and to cool, and cryogen control unit 134 is operable to individually supply to each probe 135 either a heating gas or a cooling gas.

Optionally, one or more thermal sensing probes 165 may be inserted in the vicinity of the treated organ, each thermal sensing probe 165 comprising one or more thermal sensors. One or preferably a plurality of thermal probes 165 may be connected a thermal interface box 164 using cable or wireless communication links. Signals indicative of temperature readings at probes 165 are transferred to planning unit 136 via thermal interface box 164.

System 100 may comprise one or more additional imaging apparatus 160, such as an x-ray imager, digital or film based camera, or fluoroscope. Additional imaging apparatus 160 are preferably connected to planning unit 136, providing additional sources of images of the treatment locus within the body of the patient, which images are used during planning and ablation phases of treatment. Optionally, additional apparatus 160 may be located remotely, images acquired thereby being electronically transferred to planning system 136 via communication link, or stored on removable memory media for subsequent uploading into planning system 136. Apparatus 160 might, for example, be an MRI at a remote site, used to create pre-operative images of the patient. CT cameras and functional imaging devices such as nuclear gamma camera acquiring planar images or Single Photon Emission Tomographic (SPECT) images or Positron Emission Tomograph (PET) are additional examples of additional imaging apparatus 160.

In preferred embodiments, thermal sensing probes 165 and ablation probes 135 comprise one or more echogenic surfaces, which surfaces aid in making such probes easily visible under ultrasonic imaging. Additionally or alternatively, these probes may comprise X-ray markers such as dense or opaque structures which aid in making such probes visible under x-ray imaging. It is noted that the location of a probe or other object in three-dimensional space may be deduced from two or more non-coaxial x-ray images.

In a preferred embodiment probes 135 have a shafts which comprises markings 137 visible under ultrasound or markings 132 visible under x-ray imaging (e.g. fluoroscope), which markings show visible measurements of shaft distances from distal operating tips of the probes, making it possible for an observer seeing probes 135 under an appropriate imaging modality to accurately measure the position of an operating distal tip of a probe 135 even when probe 135 is operated in cooling and its distal operating tip is not directly visible under the imaging modality because it is encased in an iceball opaque to the modality (as iceballs are opaque, for example, to ultrasound). Thus an observer of a visible portion of a shaft of such a probe 135 can calculate, based on markings 132 or 137, the position of the probe's invisible distal operating tip.

In an additional preferred embodiment system 100 comprises a vibrator 131 attachable to a probe 135 (or any other therapeutic probe of system 100). Vibrator 131 is operable to impart a high-frequency vibration to the probe 135 to which it is attached, while that probe is inserted in a patient, and even while the probe is operated in cooling. According to this embodiment ultrasound probes systems 126/126 include a Doppler detector 128 operable to detect a vibrating probe 128, by detecting Doppler variations in ultrasound echoes reflecting from the vibrating probe. According to this embodiment display 138 is operable to display the received ultrasound image while highlighting, within the image, echoes having a detected Doppler variation. Doppler detection of inserted probes can be particularly useful as an aid to registration of images in a common coordinate system. For example, two ultrasound images, one created by ultrasound probe 124 and another created by ultrasound probe 126, may easily be related to a common spatial coordinate system if one or preferably several inserted probes can be unambiguously identified in both images. Imparting a vibrational frequency to an inserted probe and utilizing Doppler detection to detect that probe provides an unambiguous means for automatically detecting that same probe in both images. Imparting different vibrational frequencies to a plurality of probes provides an unambiguous means for automatically detecting each of that set of vibrating probes in both images, making image registration relatively easy to accomplish algorithmically.

It is noted that functions ascribed herein to specific functional modules may be executed by other included functional modules without thereby altering essential aspects of the invention. For example, functions ascribed to interface box 164 or ultrasonic control unit 122 may be integrated into planning unit 136. Functions ascribed to planning unit 136 (e.g., display of ultrasound images) may be provided by a dedicated display associated with ultrasonic control unit 122.

In some embodiments, ultrasonic control unit 122 is a commercially available ultrasonic system equipped with matching ultrasonic probes and may comprise display, input devices, printer and visual output device. In these embodiments, planning unit 136 interfaces with ultrasonic control unit 122 at least to the extent of receiving from unit 122 ultrasonic images acquired by the ultrasonic system. Optionally, planning unit 136 may also receive from and transmit to ultrasonic control unit 122 signals indicative of various performance parameters, such as image zoom for example.

It should be noted that thermal ablation other than cryoablation may be performed using system 100 according to the current invention by replacing cryoprobes 135 with thermal probes.

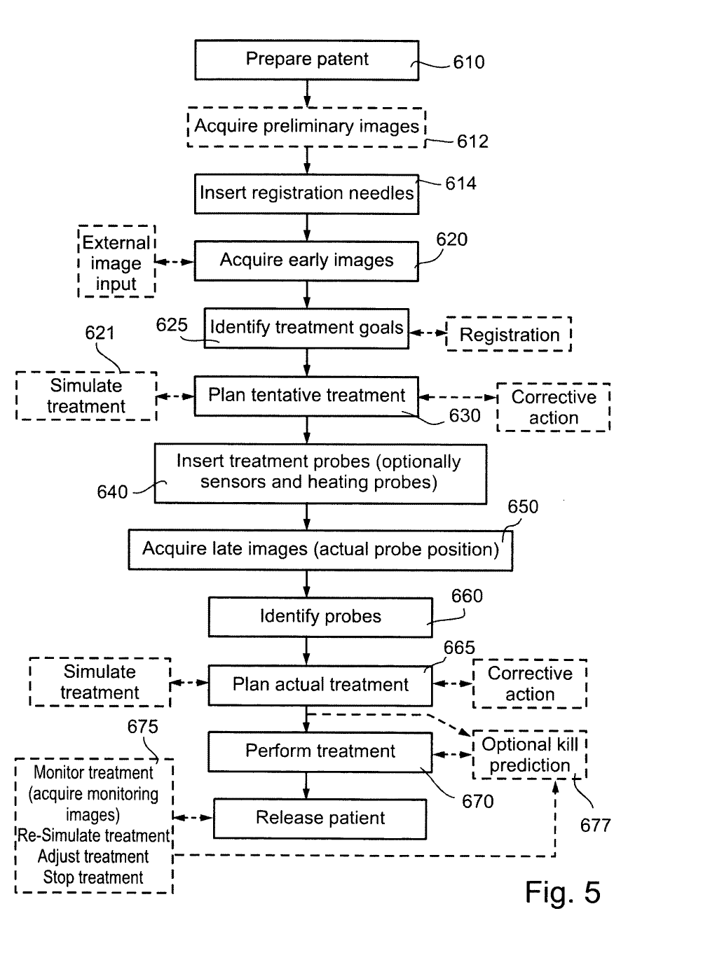

Attention is now drawn to FIG. 5, which is a simplified flowchart of a method for planning and control of a surgical intervention, according to an embodiment of the present invention. FIG. 5 details a procedure whereby system 100 acquires a first set of images of a patient (which set of images is referred to herein as “early images”), facilitates user input characterizing which tissues are to be ablated and which preserved, optionally receives user definitions of a cryoprobe setup (a set of cryoprobe insertion positions and cryoprobe operating parameters), calculates a predicted outcome of use of the setup and displays that outcome to a user, optionally plans (i.e. recommends) a cryoprobe setup, inserts cryoprobes according to a user-defined setup plan or a system-recommended setup plan, or enables and optionally assists a user to so insert a set of cryoprobes, reacquires patient images after probe placement is complete, optionally updates probe location information and user-defined treatment goals (characterization of target and non-target tissues), plans a treatment or allows a user to do so, optionally calculates expected treatment outcomes, enables corrective action to change probe placements if necessary, performs treatment according to plan, while monitoring, comparing real-time situations to planned outcomes and to user-defined treatment goals, and optionally adjusts treatment parameters during treatment and stops treatment according to plan or when required in response to a detected difference between planned and real outcomes. These procedures will now be discussed in detail.

Step 610 comprises preparing a patient for a surgical intervention, including positioning him appropriately with respect to components of system 100. Patient preparation typically comprises sedation (general or local anesthesia), attaching both patient and mechanical components of frame 158 to a bed, thereby fixing spatial relationships of patient and frame 158 and preventing motion and loss of registration, and positioning plate 150 (and/or a servomechanical device for inserting cryoprobes) with respect to the patient.

Patient preparation may also include insertion of rectal ultrasound probe 124. In preferred embodiments, rectal ultrasound probe 124 may be accompanied by a rectal warming mechanism.

At optional step 612 preliminary images may be taken, using ultrasound probe 124 or other imaging modalities.

Preliminary images taken at step 612 serve for (optional) insertion of one or more marking probes 127 at step 614. Marking probes (also referred to herein as “registration needles” may be inserted into the target organ or into other structures in the vicinity of the target organ. Marking probes 127 are probes which are visible under ultrasound or another imaging modality, are easily identified within at least some early image. Marking probes 127 are inserted in known positions with respect to frame 158. For example, marking probes 127 may be inserted in a known aperture of a probe-guide template such as is taught by Schatzberger. Marking probes 127, being echogenic, are visible in early images, and may therefore serve to enable and facilitate registration of early images with reference frame 158.

Marking probes 127 are characterized by their visibility under the imaging modality in use. Thus, they may be simple echogenic probes with no other function. Alternatively, marking probes 127 may be cryoprobes 133, thermal sensors, or other functional probes with echogenic features (or radio-opacity, or similar characteristics of visibility under the imaging modality in use. According to a recommended mode of use, a cryoprobe 133 is used as a marking probe 127, and that cryoprobe 133, after being inserted into a target, is briefly operated in cooling, causing tissues of the target to freeze and consequently to adhere to cryoprobe 133, fixing cryoprobe 133 into a position from which it cannot be dislodged during subsequent phases of treatment, and in particular during insertion of additional treatment probes into the target.

Marking probes 127 are preferably left inserted until after therapeutic treatment probes 133 have been inserted, to further aid registration of early images with late images, as described below.

Step 620 comprises acquiring what will be called herein “early images” of the patient directed towards the locus of the intended intervention and its immediate environment within the patient's body. Early images may comprise a plurality of Two Dimensional (2-D) images, and may be used to form a three dimensional image or three-dimensional model of the site, utilizing modeling techniques well known in the art. Early images may combine information from plurality of sources, including internal ultrasonic probe 124, external ultrasonic probe 126, various imaging apparatus 160.

In a preferred embodiment, early images are presented to a user as one or more two-dimensional image slices of a target site, such “slices” being acquired directly from an imaging modality or reconstructed from a 3-D model constructed from information provided by imaging modalities. Thus, one or more early image “slices” may serve as a basis for treatment planning, as described below.

At step 225, treatment goals are identified.

In preferred embodiments, early images are presented to a user, who annotates one or more of those images by identifying, on the image, anatomical boundaries such as boundaries of tissues to be ablated or tissues to be protected.

Optionally, user identification on early images of marking probes 127 and/or anatomical features visible in the images, may serve to enable or facilitate completion of processes of registration of ultrasound images, other pre-surgical or real-time images, probe insertion aid 150 (e.g. Schatzberger template), servomechanical probe aids, and various other aspects and features of system 100, with respect to common frame of reference 158.

Additionally, step 625, identification of treatment goals, comprises identifying, in the context of frame 158 and its common set of spatial coordinates, tissues which it is desired to destroy by cryoablation. In most cases it will also be necessary or desirable to identify tissues desired to be preserved from damage during the cryoablation process. In preferred embodiments, early images are presented to a user, who annotates one or more of those images by identifying, on the image, anatomical boundaries such as boundaries of tissues to be ablated or tissues to be protected. Optionally, some or all of the process of so characterizing tissues may be done algorithmically by image analysis, yet in a preferred procedure, algorithmic characterizations of tissues, if supplied, are presented to a user for his approval or amendment.

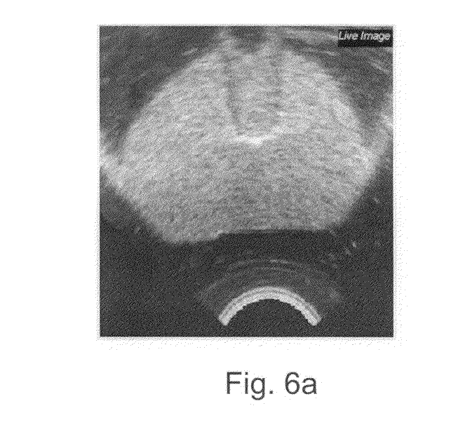

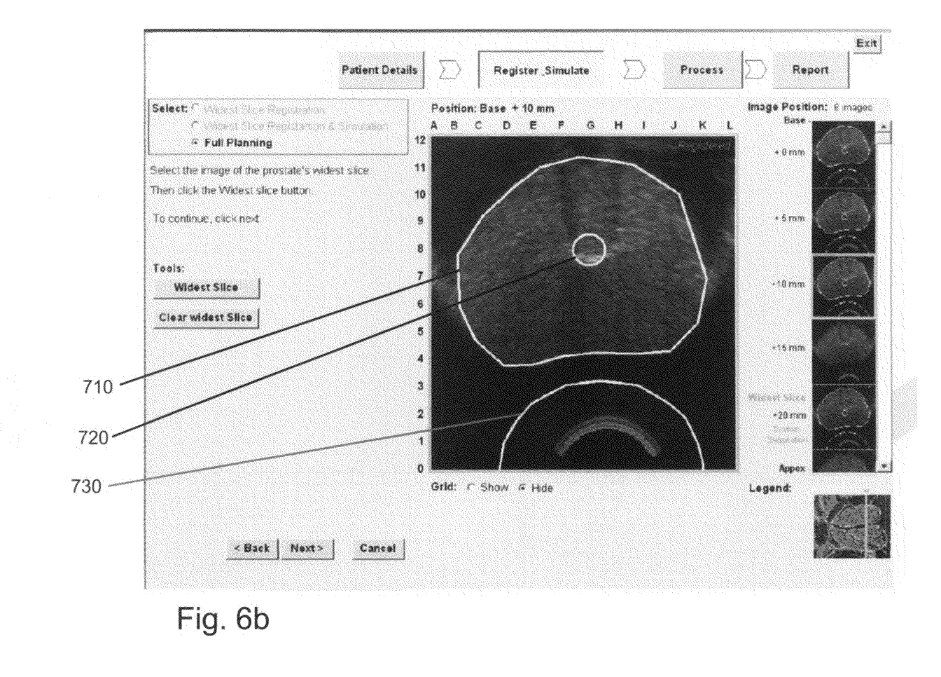

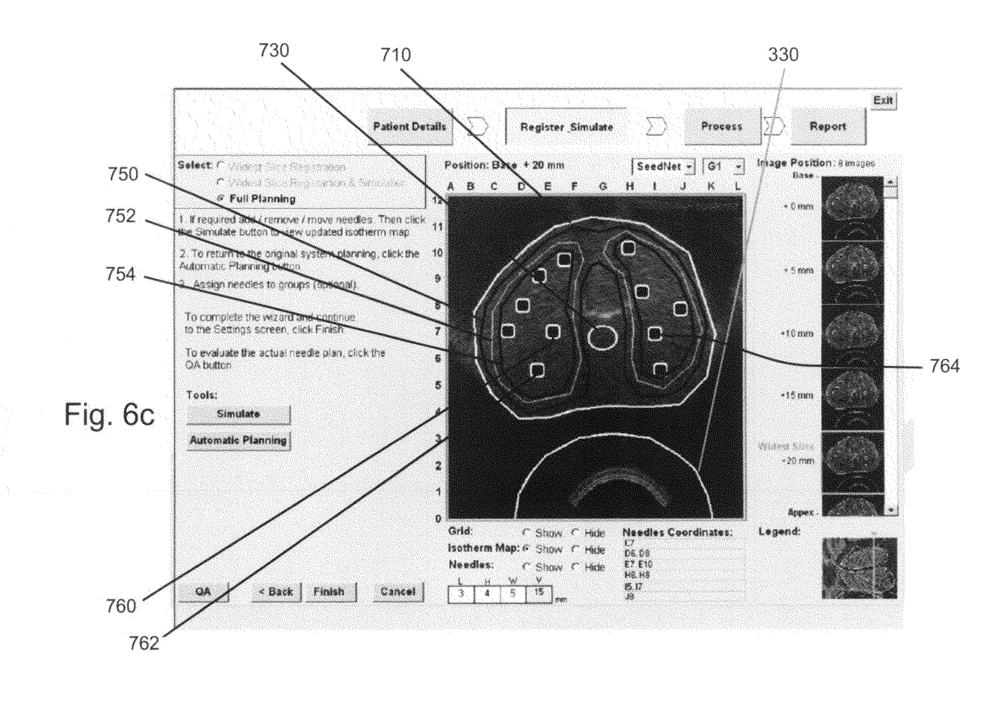

Attention is here drawn to FIGS. 6a and 6b, which illustrate this process. FIG. 6a is a raw ultrasound image of a prostate and its vicinity. FIG. 6b is an example of an annotated version of FIG. 6a, which version has been annotated according to an embodiment of the present invention. Markings on FIG. 6b indication positions of organ boundaries: as may be seen from the Figure, boundaries of a prostate 710, a urethra 720, and a rectal wall 730 are overlaid on this early image.

Identification and localization of boundaries of organs and lesions, and optionally identification and localization of other anatomical features useful for registering images or for other purposes, may be done by a user, by an automated system, or by a combination of an automated system supplying suggestions which are then accepted, rejected, or modified by a user. Image interpretation by algorithmic analysis is well known in the art. Success of any particular algorithmic approach will of course depend on the quality of the algorithm, the nature and quality of the early images being analyzed, and the degree of certainty of determination required by the clinical context. It seems probable that in many clinical contexts, particularly those in which questions of which tissues to kill are at issue, user supervision of the decision-making process, at least, will required for some time to come.

Accordingly, preferred embodiments of the present invention include features designed to facilitate tissue characterization by a user. As may be seen in FIG. 6b, an early image is preferably presented to a user in a familiar Windows-like graphical context, and the user is supplied with drawing tools of various sorts to facilitate his applying graphical marking directly to the presented image.

Thus, the step of establishing a virtual space map of a segment of a patient's body comprises the step presenting to a user at least one image that portion of the body, the image gleaned from an imaging modality, and receiving input from user, the input serving to identifying anatomical features present in that portion of the body marked by the user on the presented image.

To assist the user in this process, system 100 may utilize edge detection algorithms and curve-fitting algorithms to provide smoothed curve markers approximating detected edges of the image, as proposed boundaries. In some contexts system-proposed boundaries can be used as supplied, but in preferred embodiments users are invited to approve, disapprove, or modify boundaries proposed by the system.

Thus, for example, in treating a prostate, boundaries of the prostate will be identified in each of a sequence of ultrasonic ‘slice’ images. The plurality of 2-D boundaries thus input may be used as described above to create a 3-D model of the prostate. Structures internal to the organ, such as the urethra, and structures adjacent to it, such as the Neurovascular bundle or rectal wall, will be marked as well.

Geometric restrictions such as convexity smoothness of the resulted model may be imposed.

User marking of structure boundaries may be assisted by various facilitating features. For example, geometric restrictions such as convexity of the resulted model may be imposed. Initial ‘guesses’ by the system may be modified by ordinary graphics tools such as enlarge/reduce, shift, rotate, deform, etc. Optional initial guesses may be provided according to user-selectable preferences (e.g. “normal”, “enlarged”, “short”, “long”, etc.) Features may be provided enabling the user to point to a position on a boundary marker, hold down a mouse button and “pull” the boundary, where “spline” functions move the boundary marker under constraint of smoothness. Functions may be offered, enabling a user to mark several points and automatically generate a smooth curve connecting them. In other words, a variety of graphical manipulation options may be offered to simplify and otherwise facilitate the process of user marking of anatomical boundaries. Of course, once boundaries have been graphically marked, system software translates the graphical marks on screen images into coordinates in the virtual 3-D space of frame 158, for use in relating to and interpreting subsequently received images, optionally for use in controlling cryoprobe insertions, and optionally for evaluating and controlling ablation procedures.

In some embodiments, automatic image processing software determines the structure boundaries. For example, a fitting algorithm may use an initial guess and iteratively optimize the boundaries' shape to achieve best fit to the acquired image. Optionally the user may assist the software by choosing the initial guess or modifying it as described above to approximate the organ's shape before the fitting algorithm starts. Optionally, once the boundaries are determined, the user may accept or modify them or optionally re-acquire the image and re-start the process.

In preferred embodiments, an additional marking facilitation feature is supplied. A database of feature markers 111 (shown in FIG. 4) may be maintained within a memory 112 of a feature matching module 113, which feature markers are characterized according to measured characteristics of patient types or organ types, and by general characteristics. Feature-matching module 113 can then use inputted or discovered information characterizing a particular patient or organ to search database 111 for a feature matching marking likely to fit a feature of a particular patient and organ by virtue of known similarities between actual patient and searched database entry. The found feature marker can then be superimposed over the early image on a trail basis, be accepted or rejected by the user, or be moved or graphically modified by the user to enhance the ‘fit’ between marker and anatomical boundary visible to the user on the early image. In other words, system 100 assists a user to identify an anatomical feature by providing, superimposed on a early image on a trial basis, a feature marker derived from a collection of feature markers expected to resemble a anatomical features of that expected type (e.g. the anterior wall of a prostate), and by enabling the user to use the presented feature marker to mark an anatomical feature in the presented image. Preferably, the feature marker presented by the system is selected from collection 111 of feature markers according to similarities between physical or symptomatic characteristics of the patient and indexed characteristics describing feature markers of collection 111. In an optional version of this embodiment, feature marker collection 111 may derive from a collection of marked features of actual patients, and physical or symptomatic similarities between actual patient and historical patient may be used as a function of database selection.

To further facilitate user marking of anatomical features in images, system 100 may accept input from a user with respect to one early image, then reproduce that user input in a similar position on another early image, thereby enabling the user to identify an anatomical feature present in said late image by modifying the reproduced input with respect to the late image. Thus, anatomical boundaries marked on one ‘slice’ 2-D image of early images (e.g. an ultrasound slice image taken at a particular depth of penetration of an ultrasound probe 124) can be tentatively transferred to an image of another ‘slice’ 2-D image (e.g. an ultrasound slice image taken at another depth of penetration of ultrasound probe 124), there to be accepted, rejected, moved or modified by a user as described above.

In the case of marking a sequence of ‘slice’ images representing a sequence of ultrasound images taken at known distances one from another, interpolation between marked boundaries on two images may be used to propose approximate tentative boundaries on a third image between the two. Thus one might, for example, mark prostate boundaries on a first (e.g. most shallow ultrasound probe penetration) slice image showing a prostate, on a last (e.g. deepest ultrasound probe penetration) slice image showing the prostate, and on that slice image showing the broadest prostate image. Then, by matching a curve (in the depth dimension) to portions of marked boundaries on first, last and largest slices, good approximations of boundaries on intervening slices may be achieved. Each time a user approves or modifies a border on an intervening slice, interpolation of the other as-yet-unapproved slices may be updated using the collection of user-confirmed boundaries in the user-examined slices, resulting in progressively better and better fit between system-proposed boundaries and actual boundaries, and thereby facilitating user input of full organ boundary information.

Along with organ boundary marking, additional information may be supplied. For example, if in prostate surgery a urethral warming catheter or rectal warming device is to be used, an operator might input this fact to system 100, optionally specifying operating temperatures of these devices, which information would be used during various calculations to be described hereinbelow.

In prior-art systems treatment goals are designated in ‘black and white’ fashion, with any given tissue being designated as marked for destruction, marked for preservation, or unmarked. However, according to preferred embodiments of the present invention, tissues are characterized according to a graduated scale, which scale which extends from characterizing tissues as being highly desirable to be destroyed to characterizing tissues as being highly desirable to be preserved, with a plurality of optional gradations therebetween. User marking of image regions according to such a graduated scale may be accomplished utilizing standard graphics tools much as described above, with the addition of standard graphics tools for ‘painting’ (i.e. characterizing) large image areas. Gradations along a ‘desirability-of-destruction’ scale may be indicated by transparent color overlays or by any similar graphic means. Users are of course expected to input such information based on sources of generalized clinical knowledge (general clinical experience), patient-relevant information sources (biopsy results, clinical test results, etc.), clinical readings and interpretation of images, etc.

In addition to user-specified scoring or weighting on a ‘desirability of destruction’ scale, similar input may in some cases be gleaned from images derived from imaging modalities under automatic or semi-automatic analysis. For example, in a preferred embodiment weighted desirability-of-destruction scores for body regions may be generated automatically or semi-automatically as a function of image intensity of pixels of an image supplied by an imaging modality (e.g. tumor scintigraphy PET scans) wherein image pixel intensity is known to be correlated with probability of malignancy.

With reference again to the treatment process described by FIG. 5, in an optional step 621 which may be practiced before or after user identification of treatment goals, a user may choose to enter a “simulation mode” in which he inputs to system 100 his selection of locations for insertion of cryoprobes and user-defined parameters for operating those probes, the locations being defined with respect to a early image registered with frame 158. Planning unit 136 then uses this input information as input to a thermodynamic modeling system operable to simulate effects of the defined treatment over time, and to predict temperature outcomes throughout the treatment locus. Suitable simulation software is available commercially, for example from Noran Engineering Inc., of Westminster, Calif. (http://www.nenastran.com).

In preferred embodiments, users then view the simulated treatment outcomes. Treatment outcomes may be presented in the form of temperature isotherms imposed on early images, or graphed over time for selected user-designated positions, or may indeed be presented in the form of an animated ‘movie’ of treatment outcome situations showing isotherm progression over time in sequential images over all or part of a planned span of treatment. Such animated presentations can of course be run at a speed and temporal direction which is under user control. Users may also control the frame of reference of any of the displays mentioned above: since outcome displays derive information from calculated values in a three-dimensional model in virtual space, and may be displayed as still or animated images of a selected plane within that space, position and orientation of the plane to be displayed may also be put under user control. Additionally, using methods well known in the art, the four-dimensional information set (three physical dimensions plus time) may in fact be subject to user-controlled animated displays of any two of the four dimensions shown as a sequence of images of any selected two dimensions varying over a third dimension. Further additionally, using techniques well-known in the art of stereoscopic display, three-dimensional information varying over time can be displayed as a stereoscopic three-dimensional animation giving a viewer a true ‘in depth’ sensory experience of projected progression of the ablation process over time.

In a further preferred embodiment, an estimation function or table of estimated or observed clinical outcomes may be used to convert time and temperature information for each location into an estimate of tissue survival for that location, and these tissue survival estimates may be presented, for example in the form of shadings or transparent color variations imposed on early images showing expected tissue survival probabilities at selected user-designated times or at end of treatment, or, using tabular lookup methods described hereinabove with respect to prior art, show projected tissue survival percentages or probabilities at a future time. Data relating to survival of tissues of particular organic types under varying conditions of cooling over time are available in the clinical literature. Calculated survival percentages can be displayed with colors or pixel intensities or shadings of various sorts used to show projected tissue survival probabilities. Here too, an animated ‘movie’ rendition can dramatize expected treatment processes, for example relating projected tissue survival probabilities to expected iceball dimensions with respect to a given set of cryoprobe emplacements and operating parameters.

The simulation/prediction process described above may be undertaken iteratively, with the user amending his selection of probe locations, moving, adding or removing marked probe or probes.

It is noted that if step 621, simulation of treatment, is practiced after a user has identified treatment goals, then simulation 621 may further comprise a comparison of treatment goals with simulated outcomes. If weighted ‘desirability-of-destruction’ scores have been entered, an outcome display may use complementary graphics modes (e.g. color+symbol overlays) to display an image combining user-supplied tissue-preservation-desirability scores with a map of predicted tissue destruction probabilities according to a given set of designated cryoprobe positions and cryoprobe operating parameters. Alternatively and perhaps preferably, graphical or other types of feedback may be supplied to dramatize or emphasize particularly high or particularly low correlations between what a user has identified as a desirable profile of tissue destruction, and what a simulation has predicted as a profile of tissue destruction probably to be expected under a defined set of probe placements and probe operating parameters. Colors or light intensities or other graphic feedback devices may be used to display a fine-resolution map of the comparison of weighted ‘desirability-of-destruction’ scores with calculated probability of destruction scores, for example by tinting in green areas where goal status and predicted output status agree, tinting in red areas where goal status and predicted output status disagree, and using a range of colors between green and red to show intermediate degrees of status agreement.

Thus, temperature outcomes, minimum temperatures, maximum temperatures, tissue survival probabilities, user evaluations of tissue survival desirability, plots of survival desirability vs. survival probability, and various other calculable factors and combinations or comparisons of factors can be calculated and displayed, in stereoscopy or on user-selected two-dimensional planes, in still images and in animated temporal sequences, for any part of the images area and any part of the projected treatment period. In particular, in preferred embodiments the user can command a display of minimal or maximal temperatures at locations on a user-selected plane, can display expected percentage of tissue destruction at a selected treatment time on a user-selected plane, can use colors within an early (or late) image to express expected percentage of tissue destruction, or scores of desirability of tissue destruction, or a correlation between this two latter values, varying over time, at user-selected positions or on a user-selected plane. The user can display a graph of a tissue condition over time for a specific tissue location, or can produce a plot of tissue condition over time along a user-selected one-dimensional line.

Referring again to FIG. 5, at step 630 the user optionally requests, and planning unit 136 prepares, a tentative treatment plan to achieve the identified treatment goals of step 625. Optionally, the user inputs general parametric requirements and constraints, and may express preferences in terms of priority weights for use in comparisons of potential outcomes. Thus for example a given user may express a preference for speed of operation over minimization of costs as determined by number of needles or amount of cryogen expended, or may select or limit the number of probes to be used, or may specify that the treatment plan may or may not use probe pullback techniques requiring a thawing phase between freezing phases, or expressing a preference for symmetrical or unsymmetrical distributions of cryoprobes, and may specify acceptable levels of tissue destruction uncertainty (which levels will, of course, be radically different when treating a malignancy than when treating, say, BPH), may impose treatment length limitations relating, for example, to the desirability of reducing risks imposed by prolonged anesthesia, and so on. Even ‘non-medical’ constraints may be taken into account, such as cost differentials among treatments, optimization of surgeon time, and so on. In general, scores for all such aspects of treatment can be factored into a global score for each “treatment outcome”, with various factors being weighed according to a scale of relative importances preferably supplied as a default or according to a set of standard usage profiles, and further modifiable by a user.

These general conditions having been specified by a user, or default values or standard value sets being applied, planner 136 may generate a treatment plan. The treatment plan comprises a recommended set of cryoprobe insertion positions and operating parameters, and, generally speaking, may be calculated by a highly iterative process of creating a large number of tentative placement schemes using simple placement rules which serve primarily to avoid doing massive calculations on obviously useless configurations, by comparing calculated outcomes of cryoprobe placements to identify those with relatively high success rates, and then iterating through the identified placement combinations with varying probe operation parameter settings to identify the best outcomes, which are then presented to a user for approval.

The treatment plan selected by system 100 is then preferably presented to the user.

Attention is drawn to FIG. 6c, which presents the exemplary user-interface screen wherein aspects of a calculated treatment plan are presented to a user, according to an embodiment of the present invention. Predicted isotherm positions 750, 752, 754, etc., and recommended probe locations 760, 762, 764 may be easily seen in the Figure.

A system-selected treatment is preferably presented to the user together with a summary of predicted treatment outcomes and other characterizations of the plan. For example, in addition to presentation of the total plan score, the plan score may be contextually characterized in various ways. For example, the total score may be broken down into its components (partial scores as related to the various weighted criteria), may be presented in a manner showing availability or lack of availability of alternative plans with similar scores, may be presented in a normalized context enabling to compare that score to average scores of similar treatments (e.g. historical treatments, known to the system, of same organs of similar size), and so on. Optionally, rule-based characterizations may be provided, including plain-language interpretations such as, for example “Plan is within acceptable outcome range.”, or “No acceptable plan can be found if planning is restricted to the specified number of cryoprobes.”

Having been presented the system-recommended plan in its context, the user then accepts, rejects, or modifies the plan. The user may modify the plan by modifying the evaluation process (e.g. by modifying the evaluation weights given to various criteria (e.g. weight of cost of cryogen vs. weight of ‘cost’ of patient comfort). Alternatively, the user may simply manually input a new or changed cryoprobe insertion location or cryoprobe operating parameter, and re-run the evaluation simulation. Or further alternatively, the user may ask the system to present other configurations with scores close to that of the configuration first presented.

In step 640, cryoprobes and optionally monitoring probes are inserted into the tissue, preferably using the recommended locations from the tentative treatment plan. Preferably, a template 150 registered to reference frame 158 is used to guide the user to manually insert the probes. Alternatively, a semi-automatic apparatus may be used for insertion of probes into the tissue, wherein the user manually inserts the probes under guidance of probe positional sensors and feedback mechanisms. Further alternatively, a fully automatic apparatus such as robotic apparatus may be used for insertion of probes into the tissue.

In step 650, a new image or preferably a plurality of new images of the organ to be treated are acquired. Images created at this stage are referred to herein as “late images”.

Sources, methods of acquisition and methods of analysis of “late” images are similar to those of “early” images, and so will not be again presented in detail. The primary difference between late and early images lies in the fact that early images are created before a plurality of therapeutic probes and optional sensors and warmers have penetrated target tissues: late images are created after most or all therapeutic probes are inserted in the target area. It has been found that the process of inserting a plurality of therapeutic probes may move or displace or distort all or parts of an organ, which displacement risks rendering invalid calculations of probe positions and probe operating parameters which appeared optimal before probe insertion took place. Tissue resistance, probe flexibility, tolerances in guidance equipment, human error, and various other sources of insertion inaccuracies can cause actual location of inserted probes to depart significantly from those probes planned and intended locations. So, in preferred embodiments of the present invention, at step 660 late images acquired at step 650 may, if necessary, be examined algorithmically or manually, and inserted probes (and, if necessary, anatomical features) re-identified by users as required, using the methods of step 625 and other methods disclosed herein.

Once these late images are thus re-registered and actual positions of inserted probes and organ boundaries are re-identified, at step 665 a user is again preferably given opportunities to simulate treatment output under these newly defined conditions, to modify probe positions or probe operating parameters, and to request, receive, select and optionally modify system-selected treatment plans, and in general to engage in the same kinds of investigative and evaluative activities as were available in step 630, with the difference that simulations, planning runs and evaluations are now performed based on actual positions of organs and cryoprobes have been inserted and are no longer likely to further move nor likely to further cause movement or further distortion of body organs. Under these new conditions of real rather than hypothetical cryoprobe and organ placement, simulated treatment outcomes can be inspected to determine whether treatment goals will be adequately met, automated treatment planning may be optionally re-run if considered necessary or desirable, and if projected outcomes are not sufficiently successful under the new circumstances actual probes can be actually repositioned and the whole process repeated until a successful outcome is predicted.

In step 670, treatment is undertaken. Optionally, new images may continue to be acquired and treatment outcomes may continue to be evaluated throughout the ablation procedure, results may be displayed to a user to facilitate his processes of manual control of the operation by providing him with constantly updated status information and outcome predictions. Alternatively, some or all control of the process may be taken over by the evaluation software of planner 136, which can use the same evaluation procedures previously discussed to determine whether a dangerous departure from expected and/or desired tissue conditions exists or may be expected to exist, and to recognize when treatment goals have been fulfilled, shutting down ablation procedures in timely fashion when goals are about to be met. In particular, since ice-ball boundaries generally bear a known (if approximate) relationship to ablation volume boundaries, ice-ball boundaries, which are easily detected in ultrasound images, may be monitored and used for issuing alerts to users and/or for standard and/or emergency automated control of cooling temperatures, termination of cooling, etc. (The specific relationship between ice-ball boundaries and ablation volume boundaries will depend on the tissue being treated and other specifics of the treatment goals, such as the required degree of certainty of total ablation, etc.).

It is noted that planner 136 optionally relates to a large variety of input information and may select among a large collection of treatment alternatives. Cryoprobes may be of differing types, and in a same time of differing sizes and cooling capacities. Probes may be operated at less-than-maximum power levels or operated intermittently to produce moderate cooling. Probes and warmers may be used in common and in proximity one to another, to achieve fine control of the edge of an ablation volume. Power dosages can be adjusted so that regions with intermediate-level ‘need-to-preserve’ scores coincide with intermediate levels of destruction (note that this combination may or may not be desirable, depending on clinical considerations). Treatment plans can suggest numbers of probes, types of probes, locations of probes, can propose a power profile over a lapse of time, can suggest locations for warmers and locations for thermal sensors or other monitoring equipment, and can suggest clustering equipment (i.e. introducers for inserting and deploying a group of cryoprobes together) and suggest parameters for their deployment and functioning. And, for quality control, estimated treatment results can be stored and later compared to actual measured treatment results, and the fruits of that comparison can even be utilized in real-time situations to modify on-going predictions.

Attention is now drawn to FIGS. 7a, 7b, and 7c, which are simplified schematics showing differences in ablation outlines produced by synchronized cooling of probes, anti-synchronized cooling of probes, and cooling of a probe while heating a neighboring probe, according to an embodiment of the present invention.

FIGS. 7a-7c relate to interactions among cryoprobes. Cryoprobes with capacity to heat as well as to cool are well known in the art, and are widely used. FIG. 7a shows a two-dimensional cross-section of two treatment probes used with a synchronized cooling cycle. The term “synchronized cooling cycle” designates a cooling protocol where both probes extract head from tissue simultaneously, and may be optionally used in heating to promote thawing, also simultaneously. Synchronized cooling creates an iceball, and within it an ablation volume, both having a convex contour as shown in FIG. 7a. Outline 511 is the iceball that would result from cooling probe 510 in isolation, 521 is the iceball that would result from cooling probe 520 in isolation, and 530 represents the shape of an iceball that would result from the combined interaction of cooling by both probes 510 and 520 simultaneously. Tissues situated near both probes are cooled from both sources at once. The cumulative effect of cooling by both sources produces the convex shape seen in the figure.

FIG. 7c presents an iceball 570, indented because while probe 510 cools, probe 560 heats.

FIG. 7b might perhaps be though of as representing the result of combining the situation presented in FIG. 7a with bilateral examples of the kind of cooling and heating presented in FIG. 7c. FIG. 7b presents an iceball 540 with concave indentations. Iceball 540 is produced when probe 510 is used to heat while probe 520 is used to cool, followed by probe 520 being used to heat while probe 520 is used to cool. This scheduling pattern is referred to herein as “anti-synchronized”. If cycle alteration is sufficiently slow, anti-synchronized cooling of probes 510 and 520 produces an iceball with concave indentations, as shown in FIG. 5b.

Temperature distributions and tissue damage distributions resulting from anti-synchronized cooling can be calculated using standard thermal diffusion models. Preferred embodiments of system 100 are designed to include the possibility of anti-synchronized cooling among options experimentally used during ‘best-fit’ searches of planner 136. Thus, system 100 is operable to plan and execute anti-synchronous cooling, and to identify and correctly respond to situations where anti-synchronous cooling would be particularly useful. These include cases where a small object requiring protection, a nerve bundle for example, is positioned near an object requiring full ablation. Placing cryoprobes appropriately and cooling them anti-synchronously could be used to create a concavity in the ablation volume, which concavity might be positioned so as to limit damage to the delicate small object while affecting full ablation of the large object. As stated above, in a preferred embodiment of system 100 cryogen control unit 134 is operable to selectively supply heating gas or cooling gas to each cryoprobe 135 individually, and thus is enabled to engender anti-synchronous cooling among selected cryoprobes 135 when commanded to do so by planner 136.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An ultrasound system for use during surgery, comprising
(a) a first ultrasound probe;
(b) a second ultrasound probe;
(c) an image registration system operable to register, in a common coordinate system, information gleaned from operation of said first probe and information gleaned from operation of said second probe.

2. The system of claim 1, further comprising
(d) an image display system operable to display an image which comprises information gleaned from said first probe and information gleaned from operation of said second probe.

3. The system of claim 1, further comprising a position sensor operable to report a position of at least one of said first and second ultrasound probes.

4. The system of claim 1, further comprising an echogenic probe insertable in a body and easily visible under ultrasound imaging.

5. The system of claim 1, further comprising a motorized probe positioner operable to respond to a positioning command by positioning at least one of said first and second ultrasound probes at a position designated by said command.

6-13. (canceled)

14. A method for ultrasound imaging of a target within a body of a patient, comprising:
(a) using a first ultrasound probe to image said target from a first direction and using a second ultrasound probe to image said target from a second direction; and
(b) displaying said first and said second images simultaneously to a user, thereby providing simultaneous images of said target from two different perspectives.

15. (canceled)

16. The method of claim 14, further comprising inserting into a vicinity of said target a probe having a vibrator attachment operable to vibrate said probe, and wherein at least one of said ultrasound probes comprises a Doppler detector operable to detect vibration of said vibrating probe.

17. The method of claim 14, further comprising alternating operation of said first and second ultrasound probes, thereby avoiding signal interference between said first and second probes.

18-21. (canceled)

22. A method for ultrasound imaging of a target within a body, comprising:
(a) using a first ultrasound probe in a first position to receive ultrasound echoes from said target and using a second ultrasound probe at a second position distant from said first position to receive ultrasound echoes from said target; and
(b) creating an image which comprises information received from said first ultrasound probe and information received from said second ultrasound probe.

23. The method of claim 22, further comprising alternating operation of said first and second ultrasound probes, thereby avoiding acoustical interference between said first and second probes.

24-29. (canceled)

30. A method for monitoring a cryoablation operation, comprising:
(a) inserting a cryoprobe in a body of a patient and cooling said cryoprobe, forming an ice-ball;
(b) using a first ultrasound probe positioned at a first position to image said ice-ball from a first perspective; and
(c) using a second ultrasound probe positioned at a second position to image said ice-ball from a second perspective.

31. The method of claim 30, further comprising simultaneously displaying a first image showing a view of said ice-ball from said first perspective and a second image showing a view of said ice-ball from said second perspective.

32. The method of claim 30, further comprising creating and displaying a composite image comprising information received from said first ultrasound probe and also comprising information received from said second cryoprobe.

33. The method of claim 30, wherein said first ultrasound probe is operated from outside a patient's body and said second ultrasound probe is inserted in a body cavity.

34-36. (canceled)

37. A system for cryoablation comprising:
(a) first and second cryoprobes, each operable to cool to cryoablation temperatures and also operable to heat;
(b) a cryogen control unit programmed to alternate between a first mode which comprises heating said first cryoprobe while cooling said second cryoprobe, and a second mode which comprises heating said second cryoprobe while cooling said first cryoprobe.

38. (canceled)

39. A method of cryoablation which comprises alternating a first mode which comprises cooling a first cryoprobe while heating a second cryoprobe with a second mode which comprises heating said first cryoprobe while cooling said second cryoprobe.

40. A method of contouring an ablation volume comprising timing supply of cooling and heating gasses to a plurality of cryoprobes inserted in a body of a patient so as to effect anti-synchronized cooling of said cryoprobes, thereby creating an ablation volume with indented contour.

41. A surgery apparatus comprising:
(a) a probe insertable into a body of a patient;
(b) a vibrator attachable to said probe, and operable to impart a vibration to said probe while said probe is inserted in a patient;
(c) an ultrasound system which comprises a Doppler detector operable to detect said vibrating probe by detecting Doppler variations in echoes received from said probe.
(d) an image registration system operable to register in a common coordinate system a plurality of ultrasound images generated from different perspectives by recognizing, within said images, probe echoes having same Doppler variations.

42. A method for cryotreatment of an organ of a patient, comprising:
(a) using an imaging modality to produce a first image of a body portion;
(b) defining a treatment goal with respect to said first image;
(c) providing therapeutic probe positions for achieving said treatment goal;
(d) inserting therapeutic probes into a patient;
(e) using an imaging modality to produce a second images of said body portion;
(f) calculating probe operating parameters based on probe positions observable in said second image; and
(g) utilizing said inserted probes according to said calculated probe operating parameters to treat said patient.

43-47. (canceled)

48. The method of claim 42, further comprising inserting a position-marking probe visible under said imaging modality to mark a reference position in said body portion prior to production of said first images.

49-57. (canceled)

58. A method for simulation and prediction of surgical results, comprising:
(a) establishing a three-dimensional model of a segment of a body of a patient;
(b) establishing within said model planned positions and temperatures of therapeutic devices;
(c) calculating, for at least a portion of said model, a temperature distribution expected to result from use of said therapeutic devices at said planned positions and temperatures;
(d) calculating probabilities of tissue survival outcomes at said calculated temperatures;
(e) displaying said calculated probabilities.

59. (canceled)

60. The method of claim 58, wherein said establishing a three-dimensional model of a segment of a patient's body comprises presenting to a user at least one image of said body segment produced by an imaging modality, and receiving input from said user, said input serving to identify an anatomical feature present in said segment of said body and recognized by said user in said image.

61. The method of claim 60, further comprising providing to said user a graphical feature marker image expected to resemble a selected anatomical feature, for use in marking said anatomical feature on said image.

62. The method of claim 61, wherein said presented graphical feature marker is selected from a database of graphical feature markers.

63. (canceled)

64. The method of claim 60, further comprising accepting said input from a user with respect to a first image, reproducing said user input from said first image on a second image, and enabling said user to identifying an anatomical feature present in said second image by modifying said reproduced input with respect to said second image.

65. The method of claim 60, further comprising interpolating between a position of a first marker on a first image and a position of a second marker on a second image to calculate a proposed position of a third marker on a third image.

66-67. (canceled)

68. The method of claim 58, wherein said establishing a three-dimensional model of a segment of a patient's body comprises assigning to at least one tissue represented in an image a tissue-preservation-desirability score, said score being selected from a graduated scale of scores varying, over a plurality of gradations, between desirable to be destroyed and desirable to be preserved.

69-70. (canceled)

71. The method of claim 68, wherein said displaying said calculated probabilities of tissue survival further comprises displaying graphical elements correlated with tissue-preservation-desirability scores.

72-77. (canceled)

78. A method for display of calculated expected outputs of an ablation procedure, comprising
(a) calculating a sequence of temperature maps of a portion of a body over time, said calculation being based on a pre-defined set of cryoprobe position coordinates and a schedule of operating parameters of said cryoprobes over time;
(b) displaying information derived from said maps sequentially to a user.

79. A method according to claim 78, wherein said displaying comprises displaying an image sequence of kill probability.

80. A method according to claim 78, wherein said displaying comprises displaying an image sequence of ice-ball boundaries.

81. (canceled)

82. The method of claim 78, wherein said displayed maps display temperature differences as differences of image pixel color intensities.

83-85. (canceled)

86. The method of claim 78, wherein at lest one of said displayed maps represents temperatures at an intersection of a two-dimensional plane and a three-dimensional model of at least a portion of a body.

87-89. (canceled)

90. The method of claim 78, further comprising display of expected percentage of tissue destruction at a selected treatment time at a user-selected locus.

91. The method of claim 90, further comprising display wherein sub-pixel light intensities are calculated as functions of expected percentage of tissue destruction and of scores of desirability of tissue destruction.

92. (canceled)

93. The method of claim 90, further comprising user-commanded display of pixel color values calculated as function of a correlation between expected percentage of tissue destruction and scores of desirability of tissue destruction, for locations on a user-selected plane.

94. The method of claim 78, further comprising displaying a graph of a tissue condition over time for a specific tissue location.

95. A cryoprobe having a shaft comprising markings visible under an imaging modality while said cryoprobe is inserted in a patient and an operating tip of said cryoprobe is encased in an ice-ball generated by operation of said probe, said markings indicating distances of said markings from said tip.

96. The method of claim 22, further comprising inserting into a vicinity of said target a probe having a vibrator attachment operable to vibrate said probe, and wherein at least one of said ultrasound probes comprises a Doppler detector operable to detect vibration of said vibrating probe.
